SEQUENCE LISTING (11 sequences)

SEQ ID NO: 1 - protein, 10 aa, Pseudomonas aeruginosa
PGDLSTTGSP

SEQ ID NO: 2 - protein, 9 aa, Pseudomonas aeruginosa
RQLSGLFDA

SEQ ID NO: 3 - protein, 13 aa, Pseudomonas aeruginosa
RLPGDLSTTGSPA

SEQ ID NO: 4 - protein, 13 aa, Pseudomonas aeruginosa
SRQLSGLFDAGSG

SEQ ID NO: 5 - protein, 485 aa, Pseudomonas aeruginosa
MKRSFLSLAVAAVVLSGCSLIPDYQRPEAPVAAAYPQGQAYGQNTGAAAVPAADIGWREFFRDPQLQQLIGVALENNRDLRVAALNVEAFRAQYRIQRADLFPRIGVDGSGTRQRLPGDLSTTGSPAISSQYGVTLGTTAWELDLFGRLRSLRDQALEQYLATEQAQRSAQTTLVASVATAYLTLKADQAQLQLTKDTLGTYQKSFDLTQRSYDVGVASALDLRQAQTAVEGARATLAQYTRLVAQDQNALVLLLGSGIPANLPQGLGLDQTLLTEVPAGLPSDLLQRRPDILEAEHQLMAANASIGAARAAFFPSISLTANAGTMSRQLSGLFDAGSGSWLFQPSINLPIFTAGSLRASLDYAKIQKDINVAQYEKAIQTAFQEVADGLAARGTFTEQLQAQRDLVKASDEYYQLADKRYRTGVDNYLTLLDAQRSLFTAQQQLITDRLNQLTSEVNLYKALGGGWNQQTVTQQQTAKKEDPQA

SEQ ID NO: 6 - DNA, 22 bp, Artificial Sequence
  Oligonucleotide Primer
ttgcagcaactgatcggcgtgg

SEQ ID NO: 7 - DNA, 22 bp, Artificial Sequence
  Oligonucleotide Primer
cgcgacgttgatgtccttctgg

SEQ ID NO: 8 - DNA, 20 bp, Artificial Sequence
  Oligonucleotide Primer
cgggtcgccgcgctgaacgt

SEQ ID NO: 9 - DNA, 20 bp, Artificial Sequence
  Oligonucleotide Primer
cagcgcagctacgacgtcgg

SEQ ID NO: 10 - DNA, 20 bp, Artificial Sequence
  Oligonucleotide Primer
ccaggctggcacgcaggctg

SEQ ID NO: 11 - DNA, 20 bp, Artificial Sequence
  Oligonucleotide Primer
gcctggcgcaggtcgagcgc

(19) United States
(12) Patent Application Publication
(10) Pub. No.: US 2014/0341895 A1
(43) Pub. Date: Nov. 20, 2014

(54) PSEUDOMONAS AERUGINOSA OprM EPITOPES FOR USE IN DIAGNOSTICS AND THERAPEUTICS

(71) Applicant: Merck Sharp & Dohme Corp., Rahway, NJ (US)

(72) Inventors: Katherine Young, Metuchen, NJ (US); Francisco J. Dieguez, North Wales, PA (US); Ronald E. Painter, Piscataway, NJ (US)

(21) Appl. No.: 14/366,505

(22) PCT Filed: Dec. 17, 2012
(86) PCT No.: PCT/US2012/070019

§ 371 Date: Jun. 18, 2014

Related U.S. Application Data
(60) Provisional application No. 61/577,939, filed on Dec. 20, 2011.

(51) Int. Cl. C07K 14/21; G01N 33/569; C07K 16/12

(52) U.S. Cl. CPC C07K 14/21; C07K 16/1214; G01N 33/56911
USPC 424/133.1; 424/190.1; 424/139.1; 435/7.1

(57) ABSTRACT

The present invention relates to a peptide antigens derived from Pseudomonas aeruginosa outer membrane protein OprM for use in the diagnosis of Pseudomonas aeruginosa infection and/or prevention/treatment of diseases associated with Pseudomonas aeruginosa infection. Methods of inducing an immune response to Pseudomonas aeruginosa using peptide antigens derived from the OprM outer loops as well as derivatives or fragments thereof are also encompassed by the present invention. Compositions used to practice the methods of the invention are also encompassed.

### FIELD OF INVENTION

The present invention relates to peptide antigens derived from Pseudomonas aeruginosa outer membrane protein OprM for use in the diagnosis, prevention and/or treatment of Pseudomonas aeruginosa infection. Methods of inducing an immune response to Pseudomonas aeruginosa using a peptide antigen derived from OprM as well as derivatives or fragments thereof is also encompassed by the present invention.

### BACKGROUND OF THE INVENTION

Pseudomonas aeruginosa (P. aeruginosa) is an opportunistic pathogen that rarely causes disease in healthy people but is a significant problem for critically ill or immunocompromised individuals. Infection is a major problem in individuals who have cystic fibrosis, where P. aeruginosa is a causative agent in the progressive loss of lung function resulting from recurrent and chronic respiratory tract infections with the bacterium. Others at risk from P. aeruginosa infection include patients on mechanical ventilators, catheterized patients, cancer patients, organ transplant recipients and burn patients. P. aeruginosa accounts for between 10%-20% of all nosocomial infections (Cornelis P (editor) (2008). Pseudomonas: Genomics and Molecular Biology (1st ed.). Caister Academic Press).

Presently, P. aeruginosa infection can be controlled by antibiotics, particularly using a combination of drugs. However, resistance to several of the common antibiotics has been shown and is particularly problematic in intensive care units.

Information concerning P. aeruginosa polypeptide sequences has been obtained from sequencing the P. aeruginosa genome (Stover et al., 2000, Nature 406(6799):959-64 and GenBank Accession No. AE004091). P. aeruginosa encodes several transporters including the MexAB-OprM multidrug transporter. This transporter is the only efflux pump that is constitutively expressed and plays a central role in intrinsic multidrug resistance in this organism (Srikumar et al., 2000, J. Bacteriol. 182:1410-14 and Saito et al., 1999, FEMS Microbiol. Lett. 179:67-72). This transporter consists of three subunit proteins, MexA, MexB and OprM. The only subunit located on the outermembrane is OprM (Morshed et al., 1995, Biochem. Biophys. Res. Commun. 210:356-62 and Poole et al., 1993, J. Bateriol. 175:7363-72). The outer membrane protein OprM has been crystallized and its structure reported in Akama et al., 2004, J. Biol. Chem. 279:52816-9. The structure shows that OprM has two outer loops exposed on the surface of the organism.

Currently, the diagnosis of a P. aeruginosa infection is based upon clinical findings, microbiological cultures and biochemical tests. Gram stains of direct smears from patients are of little or no value. In fact, the most reliable method to date is the isolation of the bacterium in pure culture and its subsequent identification by biochemical or serological methods. Conventional laboratory culture techniques involve incubating clinical samples for between about 24 to about 48 hours to allow the organisms to multiply to macroscopically detectable levels. Subculture techniques and metabolic assays are then required to distinguish P. aeruginosa from related pseudomonas and other enteric bacteria and may require an additional 24 to 48 hours. Accordingly, while the rapid and accurate diagnosis of a P. aeruginosa infection is highly desirable, it is not currently possible using existing reagents and techniques.

The references cited in the present application are not admitted to be prior art to the claimed invention.

### SUMMARY OF THE INVENTION

The present invention is directed to compositions and methods of treating and/or decreasing the likelihood of an infection by P. aeruginosa or a pathology associated with such an infection in a patient. Methods of the invention comprise administering to a patient in need thereof a composition comprising a polypeptide comprising an OprM outer loop, derivative or fragment thereof to generate a protective immune response. In another embodiment, methods of the invention comprise administering an antibody that specifically binds an OprM outer loop to impart passive immunity to a patient in need thereof.

Accordingly, in one aspect of the invention, there is provided a composition comprising an isolated polypeptide comprising an OprM outer loop, derivative or fragment thereof and a pharmaceutically acceptable carrier. Preferably, said composition is pharmaceutical composition such as a vaccine.

In a specific embodiment of the invention, one or more additional antigens are provided in the composition comprising an isolated polypeptide comprising an OprM outer loop, derivative or fragment thereof. The additional antigens that may be present include one or more additional P. aeruginosa immunogens, one or more immunogens from other Pseudomonas organisms and/or one or more immunogens from other infectious organisms.

In another aspect of the invention, there is provided a composition comprising an antibody that specifically binds to an OprM outer loop. Preferably, the antibody is a human or humanized monoclonal antibody.

The present invention is also includes methods of detecting or diagnosing a P. aeruginosa infection in a patient. Such methods comprise obtaining a biological sample from the patient and determining if an OprM outer loop is present in the sample. In one embodiment, the biological sample is contacted with an antibody that binds to an OprM outer loop and antibody binding to the biological sample indicates the presence of P. aeruginosa. The antibodies used in the detection/diagnostic methods of the present invention can be polyclonal or monoclonal. In another embodiment, the presence of nucleic acids that encode an OprM outer loop are detected in the biological sample to indicate the presence of P. aeruginosa. In another embodiment, the biological sample is contacted with an aptamer that binds to an OprM outer loop and aptamer binding to the biological sample indicates the presence of P. aeruginosa. The aptamers used in the detection/diagnostic methods of the present invention can be nucleic acid or peptide apatamers.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 shows the titers of polyclonal antibodies induced against OprM outer loop 1 (using immunogen of SEQ ID NO:3) in four different rabbits. Antigens used in the ELISA experiments were either outer loop 1 (SEQ ID NO:3) (closed symbols) or outer loop 2 (SEQ ID NO:4) (open symbols).

FIG. 2 shows the titers of polyclonal antibodies induced against OprM outer loop 2 (using immunogen of SEQ ID NO:4) in four different rabbits. Antigens used in the ELISA experiments were either outer loop 2 (SEQ ID NO:4) (closed symbols) or outer loop 1 (SEQ ID NO:3) (open symbols).

FIGS. 3A-3D show the polyclonal antibodies induced against OprM outer loop 1 (using immunogen of SEQ ID NO:3) in four different rabbits (A-D). Western blots are shown using either the pre-immune sera (lanes 1-6) or the post-immunization sera (lanes 7-12) to probe outer membrane preparations (OMP) from P. aeruginosa cell lines 5890, 5919, 6476 and 6477. Lanes are as follows: (1) molecular weight markers, (2) buffer blank, (3) OMP from 5890, (4) OMP from 5919, (5) OMP from 6476, (6) OMP from 6477, (7) molecular weight markers, (8) buffer blank, (9) OMP from 5890, (10) OMP from 5919, (11) OMP from 6476, (12) OMP from 6477. Only cell lines 5919 and 6477 express OprM.

FIGS. 4A-4D show the polyclonal antibodies induced against OprM outer loop 2 (using immunogen of SEQ ID NO:4) in four different rabbits (A-D). Western blots are shown using either the pre-immune sera (lanes 1-6) or the post-immunization sera (lanes 7-12) to probe outer membrane preparations (OMP) from P. aeruginosa cell lines 5890, 5919, 6476 and 6477. Lanes are as follows: (1) molecular weight markers, (2) buffer blank, (3) OMP from 5890, (4) OMP from 5919, (5) OMP from 6476, (6) OMP from 6477, (7) molecular weight markers, (8) buffer blank, (9) OMP from 5890, (10) OMP from 5919, (11) OMP from 6476, (12) OMP from 6477. Only cell lines 5919 and 6477 express OprM.

FIGS. 5A-5B show the ability of polyclonal antibodies induced against OprM outer loop 1 (using immunogen of SEQ ID NO:3) in rabbit #1 to bind to native OprM protein. Western blots are shown using either the (A) pre-immune sera (lanes 1-5) or (B) post-immunization sera (lanes 6-10) to probe outer membrane preparations (OMP) from P. aeruginosa cell lines 5890, 5919, 6476 and 6477. Lanes are as follows: (1) molecular weight markers, (2) OMP from 5890, (3) OMP from 5919, (4) OMP from 6476, (5) OMP from 6477, (6) molecular weight markers, (7) OMP from 5890, (8) OMP from 5919, (9) OMP from 6476, (10) OMP from 6477. Only cell lines 5919 and 6477 express OprM.

FIGS. 6A-6B show the ability of polyclonal antibodies induced against OprM outer loop 2 (using immunogen of SEQ ID NO:4) in rabbit #8 to bind to native OprM protein. Western blots are shown using either the (A) pre-immune sera (lanes 1-5) or (B) post-immunization sera (lanes 6-10) to probe outer membrane preparations (OMP) from P. aeruginosa cell lines 5890, 5919, 6476 and 6477. Lanes are as follows: (1) molecular weight markers, (2) OMP from 5890, (3) OMP from 5919, (4) OMP from 6476, (5) OMP from 6477, (6) molecular weight markers, (7) OMP from 5890, (8) OMP from 5919, (9) OMP from 6476, (10) OMP from 6477. Only cell lines 5919 and 6477 express OprM.

FIGS. 7A-7B show the ability of polyclonal antibodies induced against OprM outer loop 1 (using immunogen of SEQ ID NO:3) in rabbit #3 to bind to whole cells of P. aeruginosa. Whole cell immunostaining was conducted using the polyclonal antibody as primary antibody. P. aeruginosa cells either (A) lacking OprM expression (cell line 6476) or (B) expressing OprM (cell line 6477) were used.

FIGS. 8A-8B show the ability of polyclonal antibodies induced against OprM outer loop 2 (using immunogen of SEQ ID NO:4) in rabbit #5 to bind to whole cells of P. aeruginosa. Whole cell immunostaining was conducted using the polyclonal antibody as primary antibody. P. aeruginosa cells either (A) lacking OprM expression (cell line 6476) or (B) expressing OprM (cell line 6477) were used.

### DETAILED DESCRIPTION OF THE INVENTION

OprM is a transmembrane protein on the surface of Pseudomonas that contains two surface-exposed loops. The full length amino acid sequence of OprM showed differences in 24 clinical isolates of P. aeruginosa. However, the amino acid sequence of the outer loops were determined to be 100% identical in all 24 clinical isolates. The outer loops are immunogenic in mammals.

As used herein, the term “OprM” refers to an integral membrane protein on the surface of Pseudomonas that is a component of the MexAB-OprM multidrug transporter efflux pump along with MexA and MexB. The term encompasses a polypeptide of SEQ ID NO:5 (GenBank Accession No. AAG03816) from the PAO1 strain of P. aeruginosa or a naturally occurring allelic variant or a homolog from another P. aeruginosa strain. Examples of other strains of P. aeruginosa include CL 5673, CL 5727, CL5728, CL 5729, CL 5730, CL5731, CL 5732, CL 5733, CL 5734, CL 5736, CL 5737, CL 5739, CL 5701, CB 046, CB 391, CB 392, CB 398, CLB 24228, CLB 24388, LESB58, PA7 and UCBPP-PA14. In one embodiment, OprM is SEQ ID NO:5.

As used herein, the term “outer loop” refers to the two externally facing, surface-exposed loops of OprM. Location of the outer loops was determined based on the crystal structure of OprM (Akama et al., 2004, J. Biol. Chem., 279:52816-9). Outer loop 1 (SEQ ID NO:1) is amino acid residues 117-126 of SEQ ID NO:5. Outer loop 2 (SEQ ID NO:2) is amino acid residues 328-336 of SEQ ID NO:5.

In one embodiment, polypeptides comprising one or more OprM outer loops, derivatives or fragments thereof are used as a vaccine for the treatment and/or reducing the likelihood of P. aeruginosa infection. Methods of the invention encompass administering a composition comprising a vaccine of the invention to a patient in need thereof to induce an immune response.

As used herein, the phrase “induce an immune response” refers to the ability of a polypeptide, derivative, or fragment thereof to produce an immune response in a patient, preferably a mammal, to which it is administered, wherein the response includes, but is not limited to, the production of elements, such as antibodies, which specifically bind P. aeruginosa or said polypeptide, derivative or fragment thereof. The immune response provides a protective effect against P. aeruginosa infection, ameliorates at least one pathology associated with P. aeruginosa infection and/or reduces the likelihood that a patient will contract an P. aeruginosa infection.

As used herein, the phrase “an immunologically effective amount” refers to the amount of an immunogen that can induce a protective immune response against P. aeruginosa when administered to a patient. The amount should be sufficient to significantly reduce the likelihood or severity of a P. aeruginosa infection. Animal models known in the art can be used to assess the protective effect of administration of immunogen. Animal models include, but are not limited to, a rodent lung infection model (see, e.g., Hoffmann et al., 2005, Infect. Immun. 73:2504-14; Kukavica-Ibrulj and Levesque, 2008, Lab. Animal 42:389-412 and Kukavia-Ibrulj et al., 2008, J. Bacteriol. 190:2804-13), a murine thigh infection model (Fantin et al., 1991, Antimicrob. Agents Chemother. 35:1413-22 and Sugihara et al., 2011, Antimicrob. Agents Chemother. 55:5004-9) and rodent survival assay (Farinha et al., 1994, Infect. Immun. 62:4118-23).

In another embodiment, OprM outer loops, derivatives or fragments thereof are used as a target for generating antibodies. These antibodies can be administered to a patient for the treatment and/or reduction of the likelihood of P. aeruginosa infection due to passive immunity.

As used herein, the phase “passive immunity” refers to the transfer of active humoral immunity in the form of antibodies. Passive immunity provides immediate protective effect to the patient from the pathogen recognized by the administered antibodies and/or ameliorates at least one pathology associated with pathogen infection. However, the patient does not develop an immunological memory to the pathogen and therefore must continue to receive the administered antibodies for protection from the pathogen to persist. In preferred embodiments, monoclonal antibodies, more preferably human or humanized antibodies, are administered to a patient to confer passive immunity.

In another embodiment, presence of an OprM outer loop in a biological sample is used to detect/diagnose a P. aeruginosa infection in a patient. Method of detection of one or more OprM outer loops includes contacting the biological sample with an agent that detects either the OprM outer loop protein or the nucleic acid that encodes the OprM outer loop protein. Agents include, but are not limited to, antibodies (e.g., monoclonal or polyclonal), primers and aptamers (i.e., nucleic acid or peptide).

As used here, the term “biological sample” refers to sputum, endotracheal aspirate, bronchiolar levage fluid, respiratory tract sample, urine, blood, plasma, saliva, pus, ascites, wound exudate, peritoneal fluid, abdominal fluid and tears that have been collected from a patient.

As used herein, the term “aptamer” refers to small nucleic acid (DNA or RNA) or peptide molecules that adopt specific three-dimensional confirmations and have been selected for their ability to specifically target/bind to a molecule of interest (e.g., OprM outer loop) (Song et al., 2012, Sensors 12:612; Binning et al., 2012, Front Microbiol 3:29; Mascini et al., 2012, Angewandte Chemie Internatioal Edition 51(6):1316-32).

Embodiments also include one or more of the polypeptide immunogens or compositions thereof, described herein, or a vaccine comprising or consisting of said immunogens or compositions (i) for use in, (ii) for use as a medicament for, or (iii) for use in the preparation of a medicament for: (a) therapy (e.g., of the human body); (b) medicine; (c) inhibition of P. aeruginosa replication; (d) treatment or prophylaxis of infection by P. aeruginosa; or, (e) treatment, prophylaxis of, or delay in the onset or progression of P. aeruginosa-associated disease(s). In these uses, the polypeptide immunogens, compositions thereof, and/or vaccines comprising or consisting of said immunogens or compositions can optionally be employed in combination with one or more anti-bacterial agents (e.g., anti-bacterial compounds; combination vaccines, described infra).

### Polypeptides

The amino acid sequence of a wild type full length OprM from P. aeruginosa strain PAO1 is SEQ ID NO:5. The amino acid sequences of OprM outer loop 1 and 2 are SEQ ID NOs:1 and 2, respectively. Polypeptides comprising one or more OprM outer loops can be used in the methods of the present invention. Polypeptides comprising more than one OprM outer loop may have the outer loops adjacent to each other or separated by a linker amino acid sequence.

Polypeptides comprising an OprM outer loop that contain up to 10 amino acid residues that are adjacent to the outer loop in OprM can also be used in the method of the invention. The additional amino acid residues may be on either or both of the N- and C-termini of the outer loop. In one specific embodiments, OprM outer loop 1 can have two amino acid residues included at the N-terminus and one amino acid residue included at the C-terminus of the loop (SEQ ID NO:3). In another specific embodiment, OprM outer loop 2 can have one amino acid residue included at the N-terminus and three amino acid residues included at the C-terminus of the loop (SEQ ID NO:4).

Derivatives and fragments of the OprM outer loops may also be used in the methods of the present invention. Collectively, derivatives and fragments of SEQ ID NOs:1 and 2 are termed “altered polypeptides”.

As used herein, the term “isolated peptide” indicates a peptide that is separated from at least some of the other amino acid sequences which are present in the natural source of the peptide. Preferably, an “isolated peptide” is free of some of the amino acid sequences which naturally flank the peptide in the full length polypeptide from which the peptide is derived. For example, in various embodiments, the isolated peptide preferably includes no more than 10 amino acids, no more than 8 amino acids, no more than 6, amino acids, no more than 4 amino acids, no more than 2, amino acids or no more than 1 amino acid of the amino acid sequences which naturally flank the peptide in the full length polypeptide from which the peptide is derived.

As used herein, the terms “purified peptide” indicates the presence of such polypeptide in an environment lacking one or more other polypeptides with which it is naturally associated and/or is represented by at least about 10% of the total protein present. In different embodiments, the purified polypeptide represents at least about 50%, at least about 75%, at least about 95%, or at least about 99% of the total protein in a sample or preparation. In one embodiment, the term refers to peptides that are substantially or essentially free from components that normally accompany it in its native state. Moreover, an “purified peptide” is substantially free of other cellular material, viral material, or culture medium when produced by recombinant techniques, or substantially free of chemical precursors or other chemicals when chemically synthesized.

As used herein, the term “fragment” refers to a continuous segment of an OprM outer loop (i.e., SEQ ID NO:1 or 2) or derivatives thereof having at least 5 amino acid residues, at least 6 amino acid residues, at least 7 amino acid residues, or at least 8 amino acid residues and which is shorter than the full length OprM outer loop. Preferably, fragments will comprise at least one antigenic determinant or epitopic region. More than one fragment comprising at least one antigenic determinant or epitopic region may be fused together.

As used herein, the terms “epitope” or “antigenic determinant” refer to a site on an antigen to which an antibody and/or T cell receptor binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance.

As used herein, the term “derivative” refers to a polypeptide having one or more alterations, which can be changes in the amino acid sequence (including additions, deletions and substitution of amino acid residues) and/or chemical modifications. In general, derivatives retain or substantially retain the activity of inducing an immune response, preferably a protective immune response. In some embodiments, an OprM outer loop or a fragment thereof has been altered to a derivative of the invention such that one or more epitopes have been enhanced. Epitope enhancement improves the efficacy of a polypeptide to induce an immune response, preferable a protective immune response. Epitope enhancement can be performed using different techniques such as those involving alteration of anchor residues to improve peptide affinity for MHC molecules and those that increase the affinity of the peptide-MHC complex for a T-cell receptor (Berzofsky et al., 2001, Nature Review 1:209-219).

In some embodiments, a derivative is a polypeptide that has an amino acid sequence which differs from the base sequence from which it is derived by one or more amino acid substitutions. Amino acid substitutions may be regarded as “conservative” where an amino is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. In some embodiments, no more than 5 amino acid residues, no more than 4 amino acid residues, no more than 3 amino acid residues, no more than 2 amino acid residues, or no more than 1 amino acid residue are substituted.

In other embodiments, a derivative is a polypeptide that has an amino acid sequence which differs from the base sequence from which it is derived by having one or more amino acid deletions and/or additions in any combination. Deleted or added amino acids can be either contiguous or individual residues. In some embodiments, no more than 25 amino acid residues, nor more than 10 amino acid residues, no more than 8 amino acid residues, no more than 5 amino acid residues, no more than 3 amino acid residues, or no more than 1 amino acid residues are added. In other embodiments, no more than 5 amino acid residues, no more than 4 amino acid residues, no more than 3 amino acid residues, no more than 2 amino acid residues, or no more than 1 amino acid residue are deleted. Addition of amino acids may include fusion (either directly or via a linker) to at least one functional protein domain including, but not limited to, marker polypeptides, carrier polypeptides (including, but not limited to, KLH, OMPC, BSA, OVA, THY, tetanus toxoid, HbSAg, HBcAg, rotavirus capsid proteins, L1 protein of the human papilloma virus, diptheria toxoid CRM197 protein, flagellin and VLP especially type 6, 11 and 16), polypeptides holding adjuvant properties or polypeptides that assist in purification. Additionally, it will be appreciated that the additional amino acid residues can be derived from P. aeruginosa or an unrelated source and may produce an immune response effective against P. aeruginosa or another pathogen. In specific embodiments, amino acid residues that are normally adjacent to the OprM outer loop in the full length OprM polypeptide are added to the OprM outer loop.

In other embodiments, a derivative is a polypeptide that has an amino acid sequence which differs from the base sequence from which it is derived by having one or more chemical modifications of the protein. Chemical modifications include, but are not limited to, modification of functional groups (such as alkylation, hydroxylation, phosphatation, thiolation, carboxilation and the like), incorporation of unnatural amino acids and/or their derivatives during protein synthesis and the use of crosslinkers and other methods which impose conformational constraints on the polypeptides.

Combinations of additions, deletions, substitutions and chemical modifications can be present in the same derivative.

In specific embodiments, where a polypeptide comprises an OprM outer loop with one more amino acid residues that are adjacent to the outer loop in the full length OprM polypeptide, the adjacent amino acid sequences may contain substitutions, deletions, additions and/or chemical modifications.

Methods known in the art can be used to determine the degree of difference between an OprM outer loop (e.g., SEQ ID NOs:1 and 2) and a derivative.

In a first embodiment, sequence identity is used to determine relatedness. The percent identity is defined as the number of identical residues divided by the total number of residues and multiplied by 100. If sequences in the alignment are of different lengths (due to gaps or extensions), the length of the longest sequence will be used in the calculation, representing the value for total length. In preferred embodiments, the derivative has at least 85%, at least 90%, at least 95%, at least 97%, at least 99% sequence identity to the original sequence prior to alteration. Sequence identity can be calculated by known methods, including but not limited to those described in Computational Molecular Biology, Lesk, A. M., ed., Oxford University Press, New York, 1988; Biocomputing, Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

In a second embodiment, sequence similarity or homology is used to determine relatedness. In such an analysis, amino acids with functionally equivalent physicochemical properties (e.g., a positive charge, a negative charge or bulkiness) are included in the determination of similarity or homology. Amino acid substitutions that are regarded as conservative (where an amino is replaced with a different amino acid with broadly similar properties) are included as similar or homologous residues while non-conservative substitutions (where amino acids are replaced with amino acids of a different type) are not. In such embodiments, the derivative has at least 85%, at least 90%, at least 95%, at least 97%, at least 99% sequence similarity to the original sequence prior to alteration. Sequence similarity or homology can be calculated by known methods, including but not limited to those described in Computational Molecular Biology, Lesk, A. M., ed., Oxford University Press, New York, 1988; Biocomputing, Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

In a third embodiment, nucleic acid hybridization is used to determine relatedness. In such embodiments, the nucleic acids encoding the derivative will hybridize to nucleic acids encoding an OprM outer loop (e.g., SEQ ID NOs:1 and 2) under highly stringent conditions. Stringency of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

As used here, the phrase “high stringency” refers to conditions that: (1) employ low ionic strength and high temperature for washing, for example 0.015 M sodium chloride/0.0015 M sodium citrate, 0.1% SDS at 50° C.; (2) employ a denaturing agent, such as formamide, during hybridization for example, 50% (v/v) formamide with 0.1% BSA, 0.1% Ficoll, 0.1% polyvinylpyrrolidone, 50 mM sodium phosphate buffer at pH 6.5 with 750 mM sodium chloride/50 mM sodium citrate at 42° C.; or (3) employ 50% formamide, 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2.×SSC and at 55° C. in 50% formamide, followed by a high-stringency wash consisting of 0.1×SSC containing EDTA at 55° C.

### Polypeptide Production

The polypeptides, derivatives or fragments thereof for use in the methods of the invention can be produced recombinantly or chemically synthesized.

Polypeptides for use in the methods of the present invention can be produced by chemical synthesis using either solid-phase peptide synthesis or liquid-phase peptide synthesis (see Jones, The Chemical Synthesis of Peptides (Clarendon Press, Oxford) (1994)). In the solid-phase method, it is possible to use various commercially-available peptide synthesis apparatuses (e.g., Model MultiPep RS (Intavis AG)).

In oligomer-supported liquid phase synthesis, the growing product is attached to a large soluble polymeric group. The product from each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated upon treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used (see, e.g., Merrifield, 1964, J. Am. Chem. Soc. 96:2989-93). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, a number of which are commercially available. Following synthesis, the product may be removed from the resin. The blocking groups are removed typically by using hydrofluoric acid or trifluoromethyl sulfonic acid according to established methods (e.g., Bergot and McCurdy, 1987, Applied Biosystems Bulletin). Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high-pressure liquid chromatography (e.g., using a C18 column with 0.1% trifluoroacetic acid and acetonitrile as solvents) if the molecular weight of the peptide is less than 500 daltons. Purified peptide may be lyophilized and stored in a dry state until use. Analysis of the resulting peptides may be accomplished using the common methods of analytical high pressure liquid chromatography (HPLC) and electrospray mass spectrometry (ES-MS).

Alternatively, recombinant expression of a polypeptide requires construction of an expression vector containing a polynucleotide that encodes the polypeptide of interest (e.g., an OprM outer loop, derivative or fragment thereof). Once a polynucleotide encoding the polypeptide of interest has been obtained, the vector for the production of the polypeptide of interest may be produced by recombinant DNA technology. Thus, methods for preparing a polypeptide of interest by expressing a polynucleotide encoding said polypeptide are described herein.

Methods which are well known to those skilled in the art can be used to construct expression vectors containing the coding sequences of the polypeptide of interest and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a polypeptide of interest operably linked to a promoter.

The expression vector can be transferred to a host cell by conventional techniques and the transfected cells are then cultured by conventional techniques to produce the polypeptide of interest. Thus, the invention includes host cells containing a polynucleotide encoding a polypeptide of interest operably linked to a heterologous promoter.

A variety of host-expression vector systems may be utilized to express the polypeptides of interest. Such host-expression systems represent vehicles by which the coding sequences of interest may be produced and subsequently purified, but also represent cells which may, when transformed or transfected with the appropriate nucleotide coding sequences, express the polypeptide of interest in situ. These include but are not limited to microorganisms such as bacteria (e.g., E. coli, members of the Staphylococcus genus, such as S. aureus and S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps. fluorescens transformed with recombinant bacteriophage DNA, plasmid DNA or cosmid DNA expression vectors containing coding sequences of interest; yeast (e.g., Saccharomyces genus such as S. cerevisiae or S. pichia, members of the Pichia genus such as P. pastoris, members of the Hansenula genus such as H. polymorpha, members of the Kluyveromyces genus such as K. lactis or K. fragilis, and members of the Schizosaccharomyces genus such as S. pombe) transformed with recombinant yeast expression vectors containing coding sequences of interest; insect cell systems infected with recombinant virus expression vectors (e.g., baculovirus) containing coding sequences of interest; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences of interest; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter) and coding sequences of interest.

In addition, a host cell strain may be chosen which modulates the expression of the inserted sequences, or modifies and processes the gene product in the specific fashion desired. Eukaryotic modifications may include glycosylation and processing (e.g., cleavage) of protein products. Different host cells have characteristic and specific mechanisms for the post-translational processing and modification of proteins and gene products. Appropriate cell lines or host systems can be chosen to ensure the correct modification and processing of the foreign protein expressed. To this end, host cells which possess the cellular machinery for proper processing of the primary transcript, glycosylation, and phosphorylation of the gene product may be used. As used herein, the terms “polypeptide” or “an amino acid sequence of a polypeptide” includes polypeptides containing one or more amino acids having a structure of a post-translational modification from a host cell, such as a yeast host.

If desired, expression in a particular host can be enhanced through codon optimization. Codon optimization includes use of more preferred codons. Techniques for codon optimization in different hosts are well known in the art.

Once a polypeptide of interest has been produced, it may be purified by different methods, for example, by chromatography (e.g., ion exchange, affinity, sizing column chromatography), centrifugation, differential solubility, or by any other standard technique for the purification of proteins. Further, the polypeptides of interest may be fused or attached to heterologous polypeptide sequences described herein or otherwise known in the art to facilitate purification. Examples of such protein tags include, but are not limited to, chitin binding protein (CBP), maltose binding protein (MBP), glutathione-S-transferase (GST), poly-histidine, hemagglutinin (HA) and polyanionic amino acids.

### Determination of Immunogenicity and Protective Effect

The immunogenicity of a polypeptide comprising an OprM outer loop may be determined by methods known in the art for assaying antibody titers including, but not limited to radioimmunoassay, ELISA, western blot and ELISPOT.

Animal models can be used to assay the protective effect of an altered polypeptide when used as an immunogen or when antibodies that have been generated in response to the altered polypeptide are administered as passive immunity.

In some embodiments, animals with normal susceptibility to P. aeruginosa infection are used in the in vivo assays described infra. In other embodiments, animals to be used in the in vivo assays described infra are more susceptible to P. aeruginosa infection.

In one specific embodiment, animals are made more susceptible to P. aeruginosa infection by being subjected to immunosuppressive procedures prior to infection. Such immunosuppressive procedures include, but are not limited to, reduction of peripheral blood neutrophils by administration of cyclophosphamide (Sigma-Aldrich Co., St. Louis, Mo.). For example, mice are administered 12.5 mg/mL of cyclophosphamide in saline intraperitoneally on days −5, −2, and 0 (day of infection). These mice can be used in immunization as well as passive immunity assays, however, in preferred embodiments, these mice are used in passive immunity assays.

In another specific embodiment, animals are used in the assays that are naturally less resistant to P. aeruginosa. For example, A.BY/SnJ mice are a congenic strain developed at Jackson Laboratory (Bar Harbor, Me.) that have a lower LD₅₀ for P. aeruginosa than normal laboratory mice (Pennington et al., 1979, J. infect. Dis. 129:396-99 and Farinha et al., 1994, Infect. Immun. 62:4118-23). These mice can be used in immunization as well as passive immunity assays.

Briefly, living P. aeruginosa is suspended in saline and intaperitoneally administered to 4-week-old mice at day 0 at a dose of 1×10⁵ cfu/mouse (a lethal dose). The mice have either been administered a OprM outer loop vaccine previously or is administered one or more OprM outer loop antibodies immediately after infection. The protective activity against the infection is assessed based on survival after seven days.

In some embodiments, the living P. aeruginosa used in the assay described supra can be multidrug resistant P. aeruginosa such as strain MSC06120. Normally, MSC06120 growth inhibitory concentrations of imipenem, amikacin and ciprofloxacin are 32 μg/ml, 64 μg/ml, and >256 μg/ml, respectively. Assays are conducted to determine if OprM vaccination or administration of the OprM outer loop antibodies reduces the amount of antibiotic needed to inhibit MSC06120 growth. MSC06120 can be administered intraperitoneally at 1×10⁵ cfu/mouse (a lethal dose) on day 0. The protective activity against the infection is assessed based on survival after seven days.

The invention also extends to a method of identifying an immunoreactive derivative or fragments (collectively “altered polypeptides”) of an OprM outer loop. This method essentially comprises generating a derivative or fragment of the polypeptide, administering the altered polypeptide to a mammal; and detecting an immune response in the mammal. Such response will include production of elements which specifically bind P. aeruginosa and/or said polypeptide, derivative or fragment and/or have a protective effect against P. aeruginosa infection. Antibody titers and immunoreactivity against the native or parent polypeptide may then be determined.

### Adjuvants

Adjuvants are substances that can assist an immunogen (e.g., a polypeptide, pharmaceutical composition containing a polypeptide) in producing an immune response. Adjuvants can function by different mechanisms such as one or more of the following: increasing the antigen biologic or immunologic half-life; improving antigen delivery to antigen-presenting cells; improving antigen processing and presentation by antigen-presenting cells; and, inducing production of immunomodulatory cytokines (Vogel, Clinical Infectious Diseases 30 suppl. 3:S266-270, 2000). In one embodiment of the present invention, an adjuvant is used.

A variety of different types of adjuvants can be employed to assist in the production of an immune response. Examples of particular adjuvants include aluminum hydroxide; aluminum phosphate, aluminum hydroxyphosphate, amorphous aluminum hydroxyphosphate sulfate adjuvant (AAHSA) or other salts of aluminum; calcium phosphate; DNA CpG motifs; monophosphoryl lipid A; cholera toxin; E. coli heat-labile toxin; pertussis toxin; muramyl dipeptide; Freund's incomplete adjuvant; MF59; SAF; immunostimulatory complexes; liposomes; biodegradable microspheres; saponins; nonionic block copolymers; muramyl peptide analogues; polyphosphazene; synthetic polynucleotides; IFN-γ; IL-2; IL-12; and ISCOMS. (Vogel, Clinical Infectious Diseases 30(suppl 3):S266-270, 2000; Klein et al., 2000, Journal of Pharmaceutical Sciences 89:311-321; Rimmelzwaan et al., 2001, Vaccine 19:1180-1187; Kersten, 2003, Vaccine 21:915-920; O'Hagen, 2001, Curr. Drug Target Infect. Disord. 1:273-286.)

### Combination Therapies

A polypeptide comprising an OprM outer loop, derivative or fragment thereof and/or anti-OprM outer loop antibodies can be used alone or in combination with other immunogens to induce an immune response as well as in combination with antibiotics to treat P. aeruginosa. More than one OprM outer loop immunogen and/or antibody may be administered in combination. More than one OprM outer loop immunogens can be administered either on separate polypeptides or as a part of the same polypeptide.

In one embodiment, additional immunogens that may be present include one or more additional P. aeruginosa immunogens. Additional P. aeruginosa immunogens include, but not limited to, PA 4710 (US Patent Publication 2010/0330100), PA1698 (US Patent Publication No. 2010/0291070), type IV pilin protein (International Publication No. WO 2004/099250), PA1706 (U.S. Pat. Nos. 6,309,651 and 6,827,935), PA5158 (International Publication No. WO 2007/049770) and type III secretion protein PcrV (Baer et al., 2009, Infect. Immun. 77:1083-90 and Holder et al., 2001, Infect. Immun. 69:5908-10).

In another embodiment, additional immunogens that may be present include one or more immunogens targeting one or more other Pseudomonas organisms such as P. oryzihabitans and P. plecoglossicida.

In another embodiment, additional immunogens that may be present include one or more immunogens targeting other infectious organisms including, but not limited to, the pathogenic bacteria S. aureus, H. influenzae, M catarrhalis, N. gonorrhoeae, E. coli, S. pneumoniae.

In other embodiments, antibiotics can be used in combination with the vaccines and/or passive immunity of the present invention to treat a patient. Minimum Inhibitory Concentrations (MIC) of the one or more antibiotics used to treat P. aeruginosa can be lowered by administration of the antibiotic in combination with the vaccines and/or passive immunity of the present invention. The antibiotic that is administered in combination with the anti-OprM therapy includes, but is to limited to, an aminoglycoside antibiotic (such as tobramycin, gentamicin, netilmicin, tobramycin, kanamycin, neomycin, amikacin, arbekacin, azithromycin, streptomycin, netilmicin, paromomycin, rhodostreptomycin and apramycin), a cephalosporin antibiotic (such as ceftazidime, cefepime, cefpirome and cefoperazone), a quinalone antibiotic (such as ciprofloxacin, moxifloxacin and levofloxacin), a ureidopenicillin antibiotic (such as penicillin, pipericillin, ticarcillin, and azlocillin), a carbapenem antibiotic (such as meropenem, doripenem and imipenem), a polymyxin antibiotic (such as polymyxin B and colistin), a monobactam antibiotic (such as aztreonam), a sulfonamide antibiotic, a tetracycline antibiotic, a glycylcycline antibiotic (such as tigecycline) and a macrolide antibiotic.

### OprM Outer Loop Antibodies

A polypeptide comprising an OprM outer loop, derivative or fragment thereof can be used to generate antibodies and antibody fragments that bind to OprM or to P. aeruginosa. Such antibodies and antibody fragments can be used in polypeptide purification, P. aeruginosa diagnostics, P. aeruginosa identification and/or in therapeutic treatment of P. aeruginosa infection. In some embodiments, antibodies and/or antibody fragments thereof are administered to a patient in need thereof to provide passive immunity to P. aeruginosa.

As used herein, the term “antibody” as used herein includes monoclonal antibodies, polyclonal antibodies, multispecific antibodies (e.g. bispecific antibodies), single-chain Fvs (scFv) (including bi-specific scFvs), single chain antibodies, Fab fragments, F(ab′) fragments, disulfide-linked Fvs (sdFv), and epitope-binding fragments of any of the above, so long as they exhibit the desired biological activity. In preferred embodiments, antibodies of the invention used to provide passive immunity in a patient are monoclonal, more preferably humanized or human antibodies.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different determinants (epitopes), each monoclonal antibody is directed against a single determinant on the antigen. The monoclonal antibodies to be used in accordance with the present invention may be made by the hybridoma method first described by Kohler and Milstein, 1975, Nature 256:495, or may be made by recombinant methods, e.g., as described in U.S. Pat. No. 4,816,567. The monoclonal antibodies for use with the present invention may also be isolated from phage antibody libraries using the techniques described in Clackson el at. Nature 352: 624-628 (1991), as well as in Marks et al., J. Mol. Biol 222: 581-597 (1991).

“Humanized” forms of non-human (e.g., murine) antibodies are immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. Thus, a humanized antibody may be a recombinant protein in which the CDRs from an antibody from one species; e.g., a rodent antibody, is transferred from the heavy and light variable chains of the rodent antibody into human heavy and light variable domains. The constant domains of the antibody molecule are derived from those of a human antibody. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin (see, e.g., Yamashita et al., 2007, Cytotech. 55:55; Kipriyanov and Le Gall, 2004, Mol. Biotechnol. 26:39 and Gonzales et al., 2005, Tumour Biol. 26:31).

Completely human antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893 and WO 98/16654, each of which is incorporated herein by reference in its entirety. Human antibodies can also be produced using transgenic mice which are incapable of expressing functional endogenous immunoglobulins, but which can express human immunoglobulin genes., see, e.g., PCT publications WO 98/24893; European Patent No. 0 598 877; U.S. Pat. Nos. 5,916,771; and 5,939,598, which are incorporated by reference herein in their entireties.

In some embodiments, Fc engineered variants antibodies of the invention are also encompassed by the present invention. Such variants include antibodies or antigen binding fragments thereof which have been engineered so as to introduce mutations or substitutions in the Fc region of the antibody molecule so as to improve or modulate the effector functions of the underlying antibody molecule relative to the unmodified antibody. In general, improved effector functions refer to such activities as CDC, ADCC and antibody half life (see, e.g., U.S. Pat. Nos. 7,371,826; 7,217,797; 7,083,784; 7,317,091; and 5,624,821, each of which is incorporated herein in its entirety).

There are five classes of immunoglobulins: IgA, IgD, IgE, IgG, and IgM. The IgG and IgA classes are further divided into subclasses on the basis of relatively minor differences in the constant heavy region sequence and function, e.g., humans express the following subclasses: IgG1, IgG2, IgG3, IgG4, IgA1, and IgA2. In preferred embodiments, the antibodies of the invention are IgG1.

Proper glycosylation can be important for antibody function (Yoo et al., 2002, J. Immunol. Methods 261:1-20; Li et al., 2006, Nature Biotechnol. 24:210-215). Naturally occurring antibodies contain at least one N-linked carbohydrate attached to a heavy chain (Yoo et al., supra). Additional N-linked carbohydrates and O-linked carbohydrates may be present and may be important for antibody function Id.

Different types of host cells can be used to provide for efficient post-translational modifications including mammalian host cells and non-mammalian cells. Examples of mammalian host cells include Chinese hamster ovary (Cho), HeLa, C6, PC12, and myeloma cells (Yoo et al., supra; Persic et al., 1997, Gene 187:9-18). Non-mammalian cells can be modified to replicate human glycosylation (Li et al., 2006, Nature Biotechnol. 24:210-215). Glycoengineered Pichia pastoris is an example of such a modified non-mammalian cell (Li et al., supra).

### Patient Population

A “patient” refers to a mammal capable of being infected with P. aeruginosa. In a preferred embodiment, the patient is a human. A patient can be treated prophylactically or therapeutically. Prophylactic treatment provides sufficient protective immunity to reduce the likelihood or severity of a P. aeruginosa infection. Therapeutic treatment can be performed to reduce the severity of a P. aeruginosa infection.

Prophylactic treatment can be performed using a pharmaceutical composition containing a polypeptide, immunogen or antibody described herein. Pharmaceutical compositions can be administered to the general population or to those persons at an increased risk of P. aeruginosa infection.

Those in need of treatment include those already with an infection, as well as those prone to have an infection or in which a reduction in the likelihood of infection is desired. Persons with an increased risk of P. aeruginosa infection include cystic fibrosis patients, burn patients, hospital patients, patients undergoing surgery or post-operative patients, patients on ventilators, patients with weakened immunity (including AIDS patients, cancer patients, transplant recipients), patients facing therapy leading to a weakened immunity (e.g., undergoing chemotherapy or radiation therapy for cancer or taking immunosuppressive drugs), patients receiving foreign body implants (such a catheter or a vascular device), patients undergoing diagnostic procedures involving foreign bodies, patients on renal dialysis and persons in professions having an increased risk of burn or wound injury. As used herein, “weakened immunity” refers to an immune system that is less capable of battling infections because of an immune response that is not properly functioning or is not functioning at the level of a normal healthy adult. Examples of patients with weakened immunity are patients that are infants, young children, elderly, pregnant or a patient with a disease that affects the function of the immune system such as HIV or AIDS.

Foreign bodies used in diagnostic or therapeutic procedures include indwelling catheters or implanted polymer device. Examples of foreign body-associated P. aeruginosa infections include septicemia/endocarditis (e.g., intravascular catheters, vascular prostheses, pacemaker leads, defibrillator systems, prosthetic heart valves, and left ventricular assist devices); peritonitis (e.g., ventriculo-peritoneal cerebrospinal fluid (CSF) shunts and continuous ambulatory peritoneal dialysis catheter systems); ventriculitis (e.g., internal and external CSF shunts); and chronic polymer-associated syndromes (e.g., prosthetic joint/hip loosening, fibrous capsular contracture syndrome after mammary argumentation with silicone prosthesis and late-onset endophtalmisis after implantation of artificial intraocular lenses following cataract surgery).

Patents suffering from a disease or disorder caused by P. aeruginosa infection can be treated with the compositions of the present invention. Such disease or disorders include, but are not limited to, systemic infectious disease (i.e., septicemia, meningitis, and endocarditis), otolaryngologic infectious disease (i.e., otitis media and sinusitis), pulmonological infectious disease (i.e., pneumonia, ventilator associated pneumonia, chronic respiratory tract infection), surgical infectious disease (i.e., postoperative peritonitis and postoperative infection after radial keratotomy), opthalmological infectious disease (i.e., abscess of eyelid, dacryocystitis, conjunctivitis, corneal ulcer, corneal abscess, panophthalmitis, and orbital infection), urinary tract infections (i.e., due to catheterization), dermatitis, a soft tissue infection, bacteremia, a bone infection, a joint infection, and a gastrointestinal infection.

Non-human patients that can be infected with P. aeruginosa include cows, pigs, sheep, goats, rabbits, horses, dogs, cats, rats and mice. Treatment of non-human patients is useful in both protecting pets and livestock and evaluating the efficacy of a particular treatment.

In an embodiment, a patient is treated prophylactically in conjunction with a therapeutic or medical procedure involving a foreign body. In additional embodiments, the patient is immunized at about 2 weeks, 1 month, about 2 months or about 2-6 months prior to the procedure. In another embodiment, the patient is immunized prophylactically not in conjunction with a particular contemplated procedure. For vaccinations, boosters are delivered as needed. Additionally, patients treated prophylactically may also receive passive immunotherapy by administration of an antibody against P. aeruginosa alone or in conjunction with vaccination. Passive immunotherapy with an antibody against P. aeruginosa or vaccination can occur in conjunction with administration of one or more antibiotics used to treat P. aeruginosa.

### Pharmaceutical Compositions

Further features of the invention are compositions for treating patients with an P. aeruginosa infection and/or reducing the likelihood of an P. aeruginosa infection. In one embodiment, the composition of the present invention comprises a polypeptide comprising an OprM outer loop, derivative or fragment thereof described herein (“immunogenic agent”), either alone or in combination with one or more additional antigens, and is preferably an immunogenic composition or vaccine. In another embodiment, the composition of the present invention comprises an antibody that binds to an OprM outer loop, either alone or in combination with one or more additional antibodies, and is preferably able to provide passive immunity to a patient. Suitably, the compositions of the present invention comprise a pharmaceutically acceptable carrier.

A “pharmaceutically-acceptable carrier” is meant to mean a liquid filler, diluent or encapsulating substance that may be safely used in systemic administration. Depending upon the particular route of administration, a variety of pharmaceutically acceptable carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions including phosphate buffered saline, emulsifiers, isotonic saline, and pyrogen-free water. In particular, pharmaceutically acceptable carriers may contain different components such as a buffer, sterile water for injection, normal saline or phosphate-buffered saline, sucrose, histidine, salts and polysorbate. Terms such as “physiologically acceptable”, “diluent” or “excipient” can be used interchangeably.

The above compositions may be used as therapeutic/prophylactic vaccines or as compositions to provide passive immunity. Accordingly, the invention extends to the production of compositions containing as active ingredients one or more of the immunogenic agents or antibodies of the invention. Any suitable procedure is contemplated for producing such vaccines. Exemplary procedures include, for example, those described in New Generation Vaccines (1997, Levine et al., Marcel Dekker, Inc. New York, Basel Hong Kong), which is incorporated herein by reference.

A polypeptide comprising an OprM outer loop of the invention can be fused or attached to an immunogenic carrier. Useful carriers are well known in the art and include for example: keyhole limpet hemocyanin (KLH), thyroglobulin; albumins such as human serum albumin; toxins, toxoids or any mutant crossreactive material (CRM) of the toxin from tetanus, diptheria, pertussis, Pseudomonas, E. coli, Staphylococcus, and Streprococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a polypeptide of the invention can be coupled to a T cell epitope of a bacterial toxin, toxoid or CRM. In this regard, reference may be made to U.S. Pat. No. 5,785,973, which is incorporated herein by reference.

### Administration

A polypeptide comprising an OprM outer loop, derivative or fragment thereof (alone or in combination with one or more immunogens) or an antibody described herein can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Vaccines and/or antibodies can be administered by different routes such as subcutaneous, intramuscular, intravenous, mucosal, parenteral or transdermal. Subcutaneous and intramuscular administration can be performed using, for example, needles or jet-injectors.

In some embodiments, the vaccines and/or antibodies of the invention can be formulated in or on virus-like particles (see, e.g., International Publication Nos. WO94/20137; WO96/11272; U.S. Pat. Nos. 5,985,610; 6,599,508; 6,361,778), liposomes (see, e.g., U.S. Pat. No. 5,709,879), bacterial or yeast ghosts (empty cells with intact envelopes; see, e.g., International Publication WO 92/01791, US Publication No. 2009/0239264 and 2008/0003239, U.S. Pat. No. 6,951,756), and outer membrane vesicles or blebs (de Moraes et al., 1992, Lancet 340: 1074 and Bjune et al., 1991, Lancet 338: 1093).

The compositions described herein may be administered in a manner compatible with the dosage formulation, and in such amount as is immunogenically-effective to treat and/or reduce the likelihood of P. aeruginosa infection. The dose administered to a patient, in the context of the present invention, should be sufficient to effect a beneficial response in a patient over time such as a reduction in the level of P. aeruginosa, or to reduce the likelihood of infection by P. aeruginosa. The quantity of the immunogenic agents/antibodies to be administered are preferably determined taking into account factors well known in the art including the age, sex, weight, medical condition of the patient; the route of administration of the composition; the desired effect; and the particular compound employed. In this regard, precise amounts of the immunogenic agents/antibodies required to be administered will depend on the judgment of the practitioner. In determining the effective amount of the immunogenic agents/antibodies to be administered in the treatment or prophylaxis against P. aeruginosa, the physician may evaluate circulating plasma levels, progression of disease, and the production of anti-P. aeruginosa antibodies. In any event, suitable dosages of the immunogenic agents/antibodies of the invention may be readily determined by those of skill in the art. Such dosages may be in the order of nanograms to milligrams of the immunogenic agents/antibodies of the invention.

The vaccine and/or antibody composition can be used in multi-dose formats. It is expected that a dose for a vaccine composition would consist of the range of 1.0 μg to 1.0 mg total polypeptide. In different embodiments of the present invention, the dosage range is from 5.0 μg to 500 μg, 0.01 mg to 1.0 mg, or 0.1 mg to 1.0 mg. When more than one polypeptide is to be administered (i.e., in combination vaccines), the amount of each polypeptide is within the described ranges.

It is expected that a dose for a passive immunity composition of the invention would consist of the range of 1 μg/kg to 100 mg/kg of antibody. In different embodiments, of the present invention, the dosage range is from 1 μg/kg to 15 mg/kg, 0.05 mg/kg to about 10 mg/kg, 0.5 mg/kg to 2.0 mg/kg, or 10 mg/kg to 50 mg/kg.

The timing of doses depends upon factors well known in the art. After the initial administration one or more additional doses may be administered to maintain and/or boost antibody titers.

For combination vaccinations, each of the polypeptides can be administered together in one composition or separately in different compositions. A polypeptide comprising an OprM outer loop described herein is administered concurrently with one or more desired immunogens. The term “concurrently” is not limited to the administration of the therapeutic agents at exactly the same time, but rather it is meant that the polypeptides described herein and the other desired immunogen(s) are administered to a subject in a sequence and within a time interval such that the they can act together to provide an increased benefit than if they were administered otherwise. For example, each therapeutic agent may be administered at the same time or sequentially in any order at different points in time, however, if not administered at the same time, they should be administered sufficiently close in time so as to provide the desired therapeutic effect. Each therapeutic agent can be administered separately, in any appropriate form and by any suitable route.

### Detection and Diagnosis of Infection

While the rapid and accurate detection and/or quantitation of P. aeruginosa is highly desirable, it has been difficult to achieve in practice using conventional reagents and techniques. For example, laboratory culture techniques involve incubating samples for 24-48 hours to allow the organisms to multiply to macroscopically detectable levels. Subculture techniques and metabolic assays are then required to distinguish Pseudomonas aeruginosa from related Pseudomonas and other enteric bacteria and may require an additional 24-48 hours.

The present invention encompasses methods to detect/diagnose a P. aeruginosa infection in a patient using an agent that specifically binds to an OprM outer loop that are more rapid than presently used methods. Agents include antibodies (monoclonal or polyclonal), nucleic acid primers (e.g., for PCR or TaqMan) and aptamers (nucleic acid or peptide).

Such methods comprise obtaining a biological sample from the patient, contacting the biological sample with an agent that specifically binds to an OprM loop, incubating the agent/biological sample under conditions that allow for binding and detecting the binding of the agent to the biological sample. Detection of the agent binding to the biological sample indicates the presence of P. aeruginosa and/or P. aeruginosa infection.

As used here, the term “biological sample” refers to sputum, endotracheal aspirate, bronchiolar levage fluid, respiratory tract sample, urine, blood, plasma, saliva, pus, ascites, wound exudate, peritoneal fluid, abdominal fluid and tears that have been collected from a patient.

The agents used in the detection/diagnostic methods of the present invention binds substantially all strains of P. aeruginosa. No significant cross-reactivity with other species of Pseudomonas or with other clinically significant gram-negative or gram-positive species should be observed.

For use in diagnostic assays, the agents may be directly labeled. A wide variety of labels may be employed, such as radionuclides, fluorophores, chromophores, enzymes, enzyme substrates, enzyme cofactors, enzyme inhibitors, ligands (particularly haptens), etc. In some embodiments, the label used on the antibodies for use in the detection/diagnostic assays of the invention is a fluorophore including, but not limited to, GFP, YFP, CFP, fluorescein, rhodamine, phycoerythrin, phycocyanin, DAPI, FITC and Texas Red. Methods of labeling antibodies with fluorophores are known in the art.

In embodiments where the agent is an antibody, the antibody can be unlabeled and used in agglutination assays. In addition, unlabeled antibodies can be used in combination with other labeled antibodies (secondary antibodies) that are reactive with the antibodies of the invention.

Numerous types of immunoassays are available and are well known to those skilled in the art.

Kits can also be supplied for use with the antibodies of the invention. The kits comprise compartments containing (1) one or more antibodies capable of binding to an P. aeruginosa OprM outer loop, (2) labels, and (3) necessary reagents for providing a detectable signal. Thus, the antibody composition of the present invention may be provided, usually in a lyophilized form, either alone or in conjunction with additional antibodies specific for other antigens of P. aeruginosa. The antibodies, which may be conjugated to a label, are included in the kits with buffers, such as Tris, phosphate, carbonate, etc., stabilizers, biocides, inert proteins, e.g. bovine serum albumin, or the like. Generally, these materials will be present in less than about 5% weight based on the amount of active antibody, and usually present in total amount of at least about 0.001% weight based again on the antibody concentration. Frequently, it will be desirable to include an inert extender or excipient to dilute the active ingredients, where the excipient maybe present in from about 1% to 99% weight of the total composition. Where a second antibody capable of binding to the anti-OprM outer loop antibody is employed, this will usually be present in a separate vial. The second antibody may be conjugated to a label and formulated in a manner analogous to the antibody formulations described supra.

Other features and advantages of the present invention will become apparent from the following experimental descriptions, which describe the invention by way of example. The examples are offered by way of illustration and not byway of limitation.

### EXAMPLES

Examples are provided below to further illustrate different features of the present invention. The examples also illustrate useful methodology for practicing the invention. These examples do not limit the claimed invention.

### Example 1

### Identification of OprM Outer Loops

The similarity/conservation of the outer loops of the OprM efflux pump across a panel of 24 imipenem-resistant Pseudomonas aeruginosa clinical isolates was determined. The clinical isolates were all of strain PAO1 and were harvested from various patients at a number of different hospitals in the US as well as internationally. The samples were harvested from endotracheal aspirate, sputum, bronchial washes, urine, blood or wounds.

DNA was isolated from each clinical isolate in order to amplify and sequence the OprM outer loops from each. Briefly, cultures were grown overnight in TBS at 37° C. before centrifugation ay 13,000×G for 5 min. The pellet was resuspended in 200 μl of water, resuspended and heated to 95° C. for 10 min. Samples were allowed to cool on ice before centrifugation ay 13,000×G for 5 min. Supernatant was collected.

PCR primers (SEQ ID NOS:6 and 7) were designed that amplified the region of DNA encoding both OprM outer loop one and two in the same amplicon (˜920 base pairs) based on the crystal structure of OprM (Akama et al., 2004, J. Biol. Chem., 279:52816-9). PCR was run under the following conditions: denaturation at 94° C. for 2 min. before 35 cycles of 94° C. for 30 seconds, 60° C. for 30 seconds, 68° C. for 1 min. After the 35 cycles, samples were kept at 68° C. for 10 min. before maintenance at 4° C. PCR amplicons for each isolate were sent, in duplicate, to SeqWright (Houston, Tex.) for sequencing using primers (SEQ ID NOS:8-11) designed to provide double coverage of the DNA sequence.

Sequence analysis was performed using the Vector NTI software package (Invitrogen, Carlsbad, Calif.). ContigExpress was used to assemble multiple raw sequence data files into one amplicon. AlignX was used to compare the final amplicons (both nucleotide and amino acid) of each isolate.

Comparison of the sequence data revealed 100% amino acid sequence identity in the two outer loops of OprM in all of the clinical isolates. However, the clinical isolates were not related to each other as demonstrated by amino acid sequence differences of OprD, AmpC and AmpD (data not shown). Because of this conservation, the outer loop epitopes will recognize many strains of P. aeruginosa.

### Example 2

### Antigen Production

Using the OprM crystal structure as a guide (Akama et al., 2004, J. Biol. Chem., 279:52816-9), peptides were synthesized that included an OprM outer loop with some surrounding amino acid residues. For OprM outer loop 1, two amino acid residues were included at the N-terminus and one amino acid residue was included at the C-terminus of the loop. For OprM outer loop 2, one amino acid residue was included at the N-terminus and three amino acid residues were included at the C-terminus of the loop. The outer loop sequences with the included surrounding residues are shown in SEQ ID NOS:3 and 4.

Additionally, three amino acid residues (CKK) were added to the N-terminus of each peptide to increase its solubility and facilitate its conjugation to a keyhole limpet hemocyanin (KLH) carrier protein.

The peptides were synthesized by solid phase peptide synthesis using Fmoc-chemistry. The initial solid support was Chematrix resin. Fmoc was removed by 20% piperidine in DMF followed by washing with DMF. The amino acids were activated by HBTU/DIEA and coupled for 30 minutes until a Ninhydrin test indicated no detectable amino group. The peptides were built up with the same coupling strategy according to the desired sequence.

The peptides on solid support were cleaved by a mixture of TFA/dithioethanol/phenol/thioanisole (85:5:5:5 ratio by volume) for two hours. The peptides were precipitated in cold ether before they were collected, washed and dried to give crude material.

The crude peptides were dissolved in a mixture of acetonitrile and water, followed by passing through a reversed phase HPLC column. The desired peptide was collected in many fractions, whose purities were checked byoy analytical HPLC. All fractions of greater than 80% purity were combined and lyophilized to give final peptide powder.

KLH (5 mg) was activated by hetero-bifunctional linker for 30 minutes. The excess linker was removed by dialysis. The resulted solution was added purified peptide (5 mg) and reacted for 16 hours followed by dialysis to remove unconjugated peptide, then lyophilized to give final product.

### Example 3

### Polyclonal Antibody Production

Polyclonal antibodies were produced using each of the OprM outer loops (with surrounding amino acid residues) conjugated to KLH as immunogens. Rabbits were screened prior to antigen injection for background antibodies that recognize the OprM outer loop immunogens to be used (i.e., SEQ ID NOS:3 and 4). Pre-immune rabbit sera was assayed by ELISA for antibodies to the immunogen. Rabbits that demonstrated background antibodies that recognize the OprM outer loop immunogens were not used for immunization.

Eight NZW rabbits were immunized on days 0, 21, 42, 63, 84 and 105 subcutaneously with either KLH-conjugated OprM outer loop 1 (SEQ ID NO:3) or outer loop 2 (SEQ ID NO:4) peptides (500 μg per injection). The Day 0 injection was formulated with Titermax while all subsequent injections were formulated with Freund's incomplete adjuvant. The rabbits were bled on days 52 (5 ml serum obtained), 73 (20 ml serum obtained), 94 (20 ml serum obtained), 115 (20 ml serum obtained) and 118 (50 ml serum obtained). Their sera were screened for presence of an immune reaction to the OprM outer loops and it was determined that both of the KLH-conjugated outer loop peptides generated an immune response in the immunized rabbits.

### Example 4

### Characterization of Polyclonal Antibodies

Sera of the immunized rabbits was analyzed by two in vitro assays, ELISA and western blot analysis.

Sera was assayed by ELISA to measure the amount of anti-OprM antibodies that were induced by the immunization. Purified peptide from either outer loop 1 or outer loop 2 was plated each well of a 96-well plate. A solution of PBS+3% BSA was used for non-specific blocking and sample/control/secondary antibody dilutions. The sample serum was run against a negative control (normal sera) and a positive control (anti-ovalbumin). The signal was detected using HRP conjugated anti-rabbit secondary antibodies and ABTS peroxidase substrate system. The plates were read at a wavelength of 415 nm with a reference at 570 nm.

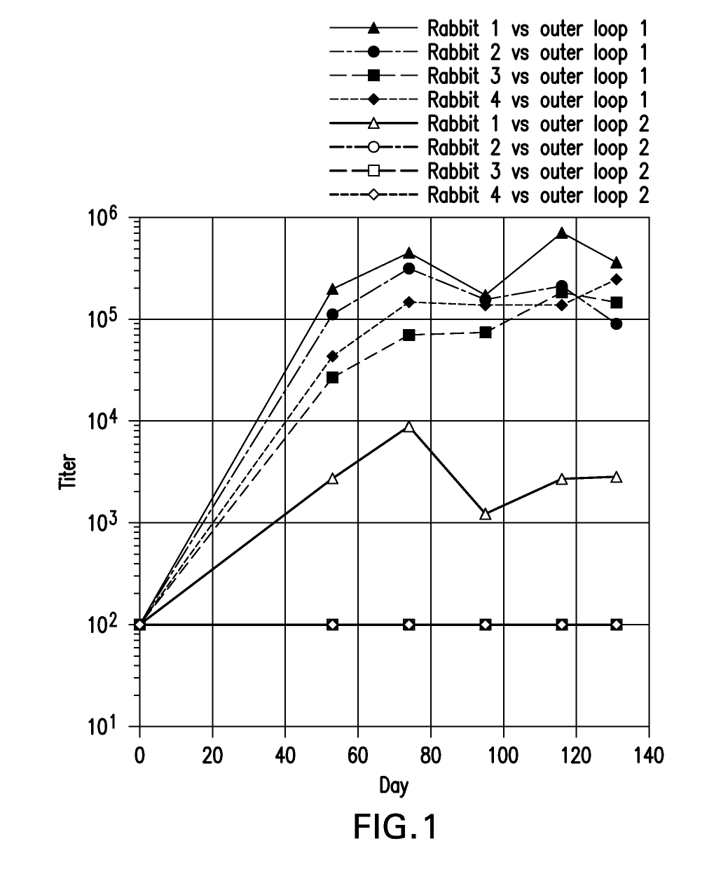

FIG. 1 shows that rabbits immunized with a peptide comprising OprM outer loop 1 generated high titer of antibodies to a peptide containing outer loop 1, SEQ ID NO:3 (closed symbols). When a peptide containing outer loop 2, SEQ ID NO:4, was used as antigen in the assay, only 1 rabbit showed a small amount of cross-reactivity (open symbols).

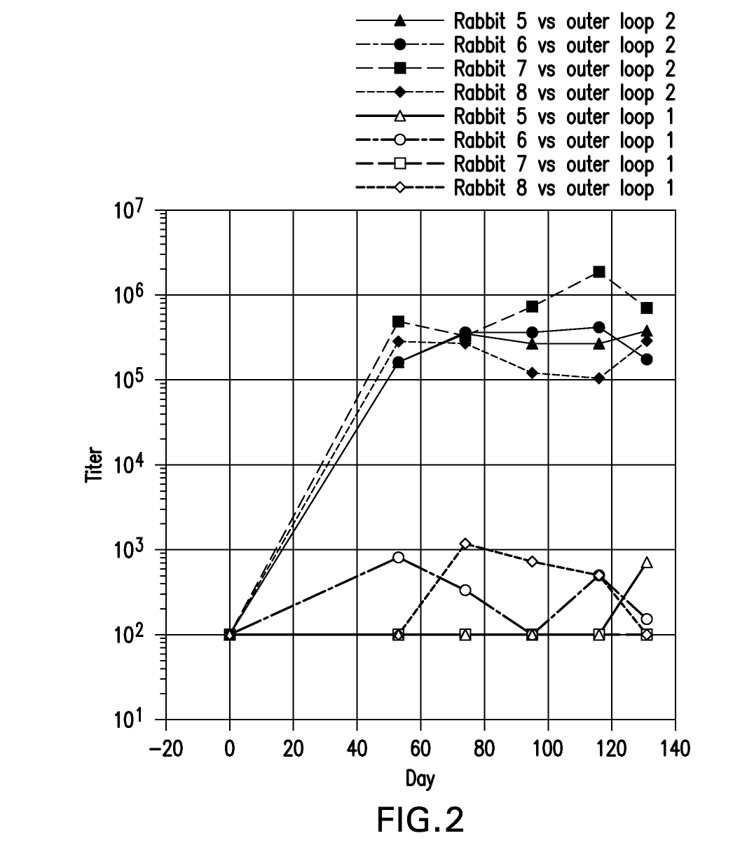

FIG. 2 shows that rabbits immunized with a peptide comprising OprM outer loop 2 generated high titer of antibodies to a peptide containing outer loop 2, SEQ ID NO:4 (closed symbols). When a peptide containing outer loop 1, SEQ ID NO:3, was used as antigen in the assay, 2 rabbits showed a small amount of cross-reactivity (open symbols).

Sera was also analyzed by Western blot to further characterize the anti-OprM polyclonal antibodies that were induced by the immunization. The antigen used in the western blot assays was an outer membrane preparation (OMP) obtained from a P. aeruginosa cell line. Sarkosyl extracts from the outer membranes of Pseudomonas strains were isolated using a procedure adapted from published methods (Yoshihara et al., 1996, FEBS Lett. 394:179-82; Hosaka et al., 1995, Antimicrob. Agents Chemother. 39:1731-5). Briefly, P. aeruginosa was grown in L-broth supplemented with 5 mM MgCl₂ for 5 h at 37° C. after a 10-fold dilution of fully grown preculture. The bacterial cells were suspended in 20 mM HEPES buffer, pH 7.4 and were burst by sonication. The membranes were pelleted by centrifugation at 100,000×g for 45 min at 15° C. and suspended in 20 mM HEPES buffer, pH 7.4. The membrane suspension was mixed with 1% (wt/vol) of N-lauroyl sarcosinate (sarkosyl) and incubated at 30° C. for 30 min. The outer membrane proteins were pelleted by centrifugation at 100,000×g for 45 min at 15° C. and washed two times with 200 mM HEPES or sodium phosphate buffer, pH 7.5.

A number of cell lines were used to prepare the outer membrane preparations. The cells were either wild type P. aeruginosa or lacked one or both of OprM and OprD. Cell lines 5919 and 6477 express OprM while cell lines 5890 and 6476 do not express OprM. (See Duncan et al., “Construction of a novel Pseudomonas aeruginosa strain panel deficient in fifty-two potential drug efflux systems” 44th Interscience conference on antimicrobial agents and chemotherapy, Oct. 30-Nov. 2, 2004, Washington, D.C. American Society of Microbiology, Washington, D.C. (2004). Abstract D-1532)

SDS slab gel electrophoresis was carried out according to the methods of Laemmli (Nature 227:680-5, 1970) as modified by O'Farrell (J. Biol. Chem. 250:4007-21, 1975). The samples were diluted to 0.5 mg/ml in SDS Boiling Buffer and loaded in wells of a 10% acrylamide slab gel such that there was 5 μg of total protein/lane. SDS slab gel electrophoresis was carried out for about 4 hours at 15 mA/gel. Molecular weight markers were obtained from Sigma Chemical Co. (St. Louis, Mo.). The gel was stained with Coomassie and dried between sheets of cellophane. After gel slab electrophoresis, the gels for blotting were placed in transfer buffer (10 mM CAPS pH 11, 10% MeOH) and transblotted onto PVDF membrane overnight at 200 mA and approximately 100 volts/two gels.

The blots were blocked for two hours in 5% Nonfat Dry Milk in TTBS saline. The blots were incubated in primary antibody overnight and rinsed 3×10 minutes in TTBS. The sera collected from the immunized rabbits was diluted 1:500,000 (in 2% Nonfat Dry Milk in TTBS) to serve as primary antibody. The blots were then placed in secondary antibody (anti-rabbit IgG-HRP) for two hours, rinsed 3×10 minutes in TTBS, treated with ECL and exposed to x-ray film for one hour.

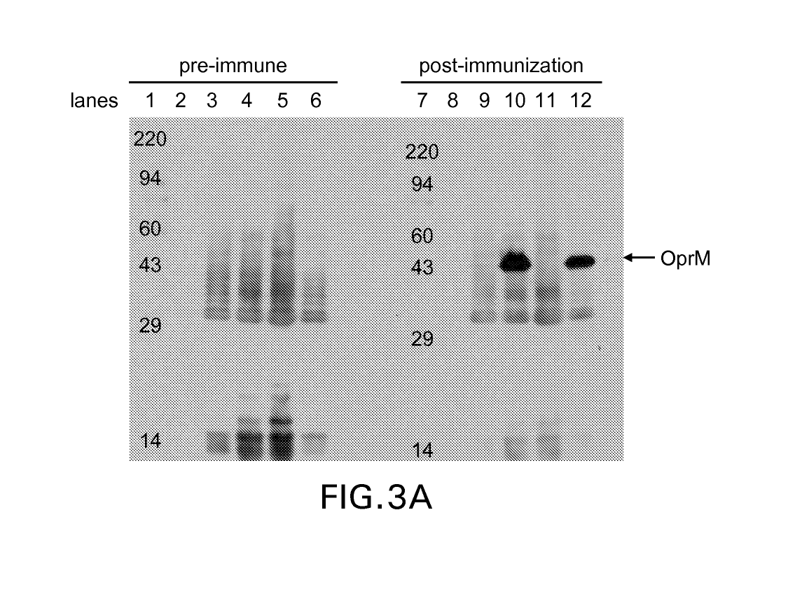

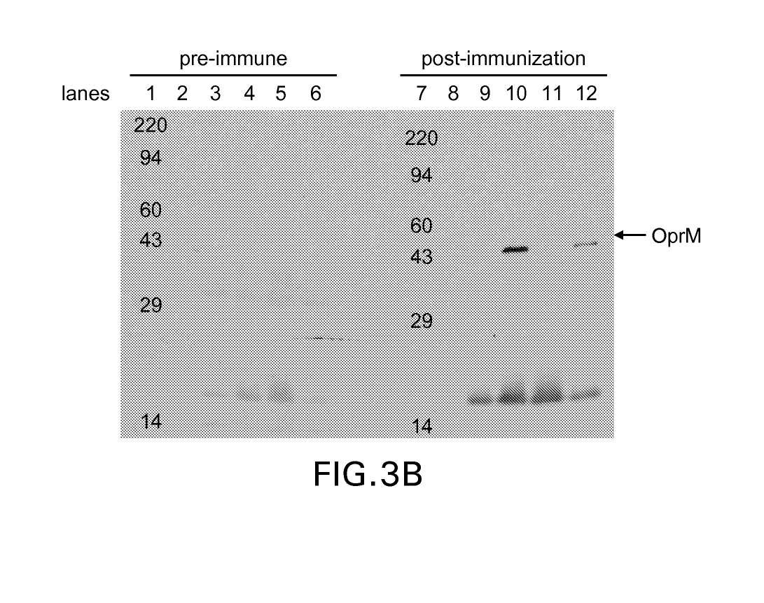

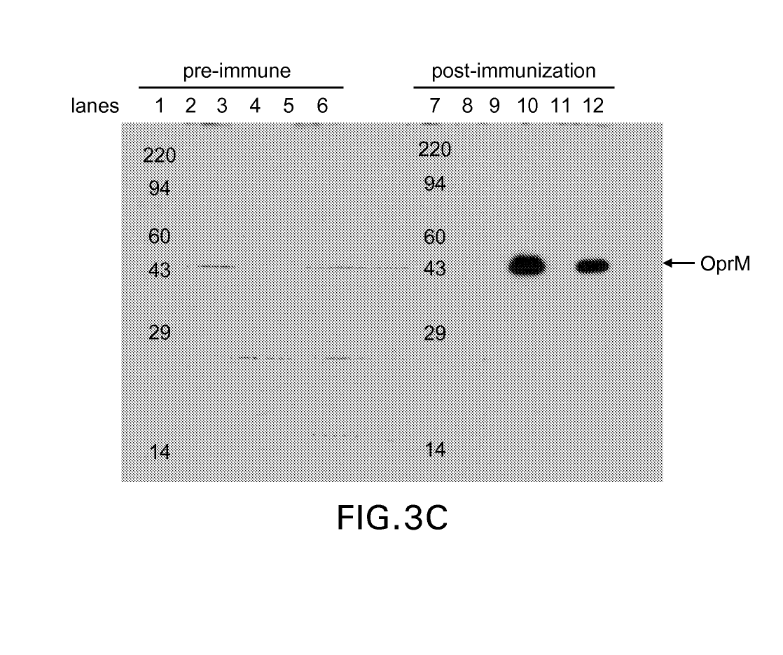

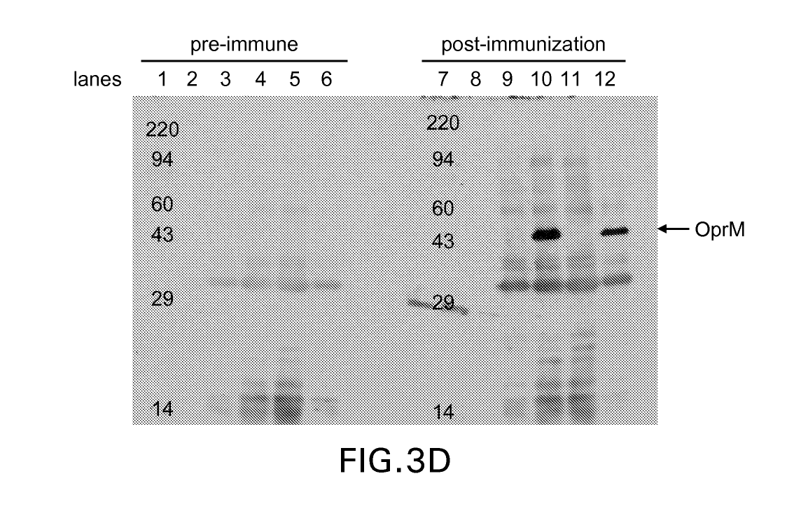

FIGS. 3A-3D show that the sera collected from four different rabbits immunized with OprM outer loop 1 (SEQ ID NO:3) recognized OprM in the two outer membrane preparations that were made from OprM-expressing cells (lanes 10 and 12). All rabbits show OprM recognition only post-immunization.

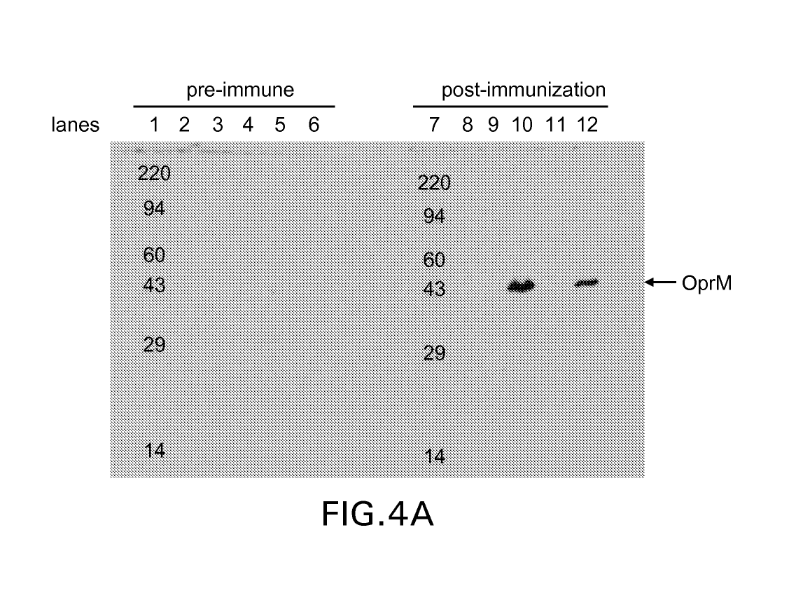

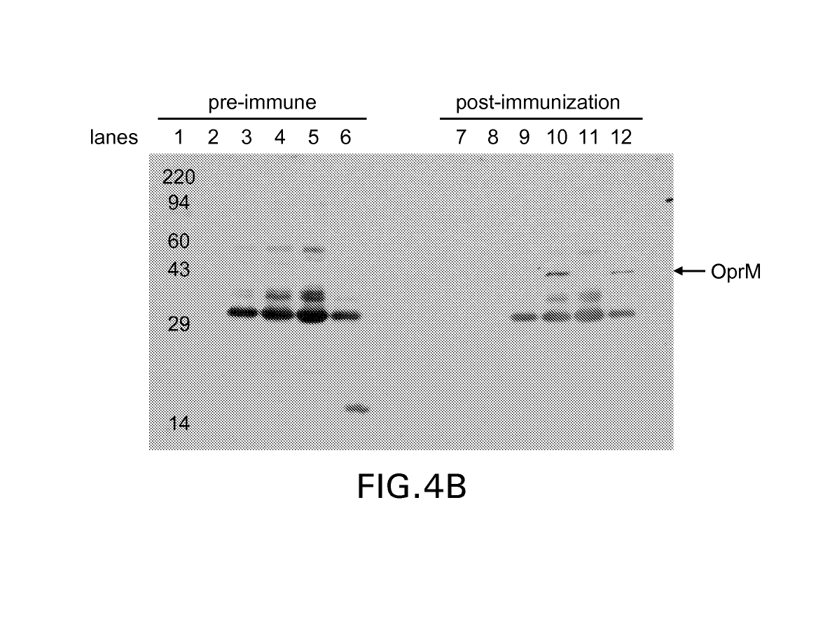

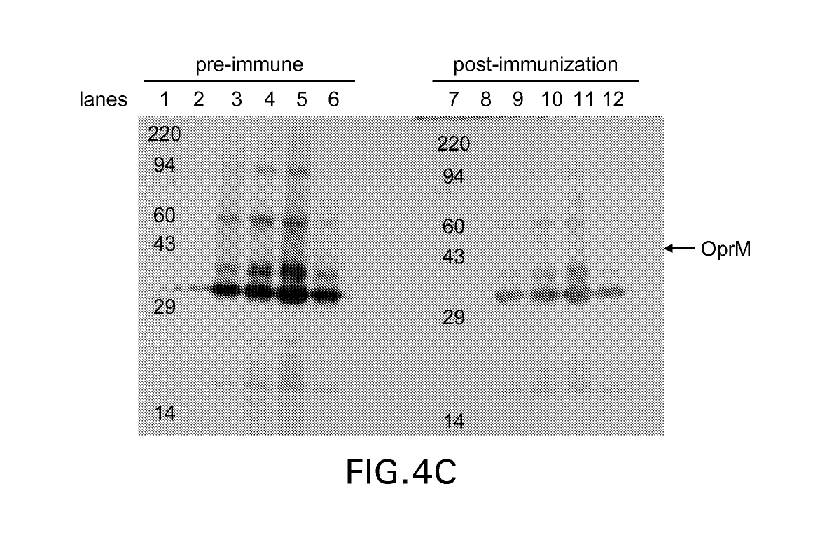

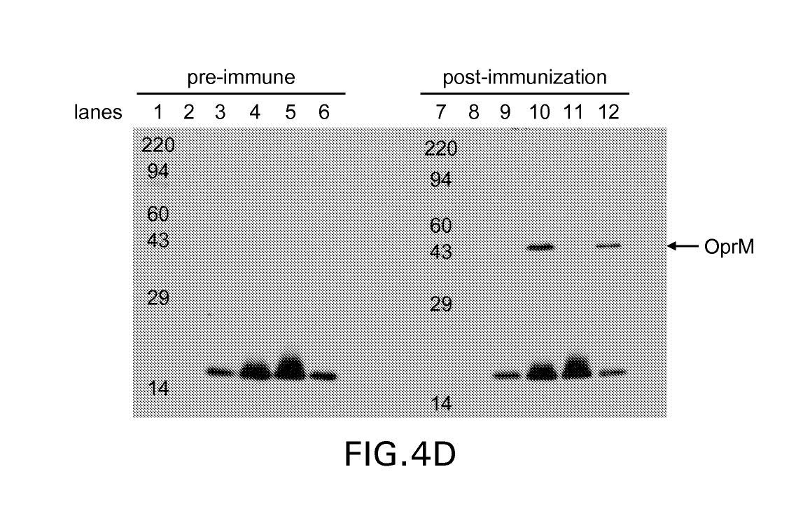

FIGS. 4A-4D show that the sera collected from four different rabbits immunized with OprM outer loop 2 (SEQ ID NO:4) recognized OprM in the two outer membrane preparations that were made from OprM-expressing cells (lanes 10 and 12). Rabbits shown in panels A, B and D show OprM recognition only post-immunization.

### Example 5

### Polyclonal Antibodies can Detect Native Protein

The polyclonal antibodies were assayed for their ability to detect native (non-dentatured) OprM by western blot and whole cell immunostaining.

Western blots were run essentially as described in Example 4 except that the outer membrane preparations were not run under reducing conditions so that the OprM protein would retain its native conformation. The samples and molecular weight markers were run on the NativePAGE Novex precast 3-12% Bis Tris (Invitrogen Cat # BN 1001BOX Lot#11102731), 1.0 mm thick minigels. The gels were run with Native PAGE Running buffer and NativePAGE Cathode Additive according to Invitrogen's “NativePAGE Novex Bis-Tris Gel System,” Version A, Jan. 20, 2006, 25-0894. Briefly, after loading the samples, the gels were run at a constant 150 V for approximately 15 minutes using Dark Cathode[1 part NativePAGE Running Buffer (Invitrogen Cat# BN2001 Lot #1114385) diluted with 1 part NativePAGE Cathode Buffer Additive (Invitrogen Cat# BN2002 Lot #1076303)]. The gel was then run with Light Cathode [10 parts part NativePAGE Running Buffer diluted with 1 part NativePAGE Cathode Buffer Additive] at constant 150 V until the dye front was at the bottom of the gel ˜70 minutes more. The gels were blotted onto PVDF membrane and OprM protein was detected as described in Example 4.

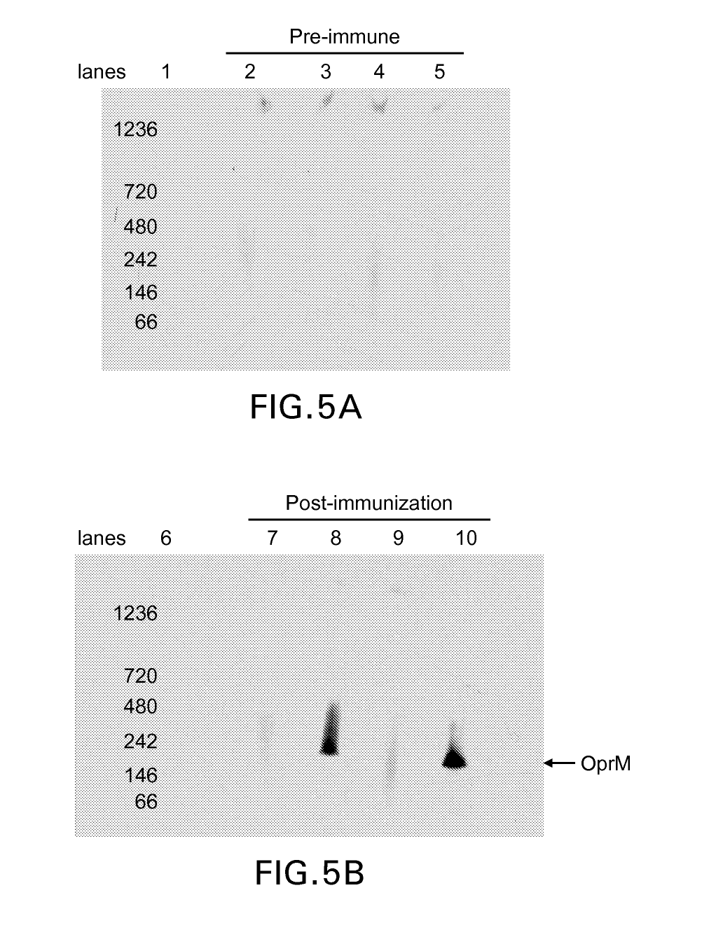

FIGS. 5A-5B show that the sera collected from rabbit #1 that had been immunized with loop 1 (SEQ ID NO:3) does recognize the native OprM (lanes 8 and 10) but no OprM signal is seen in pre-immune sera or on membrane preparations from cell lines that do not express OprM (lanes 7 and 9).

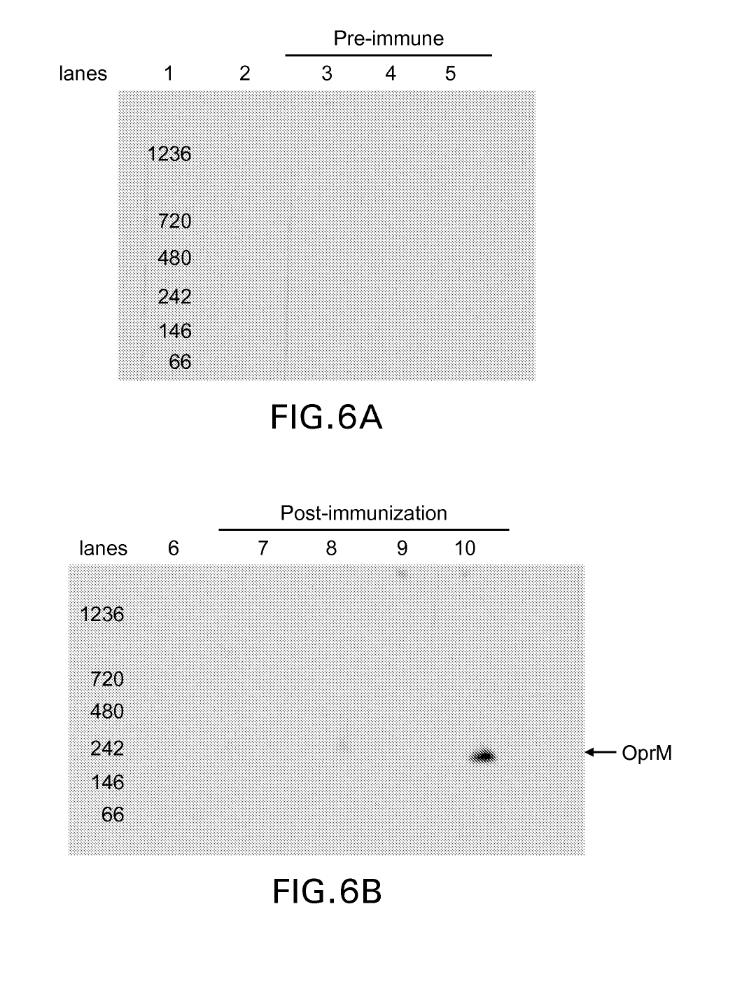

FIGS. 6A-6B show that the sera collected from rabbit #8 that had been immunized with loop 2 (SEQ ID NO:4) does recognize the native OprM (lanes 8 and 10) but no OprM signal is seen in pre-immune sera or on membrane preparations from cell lines that do not express OprM (lanes 7 and 9).

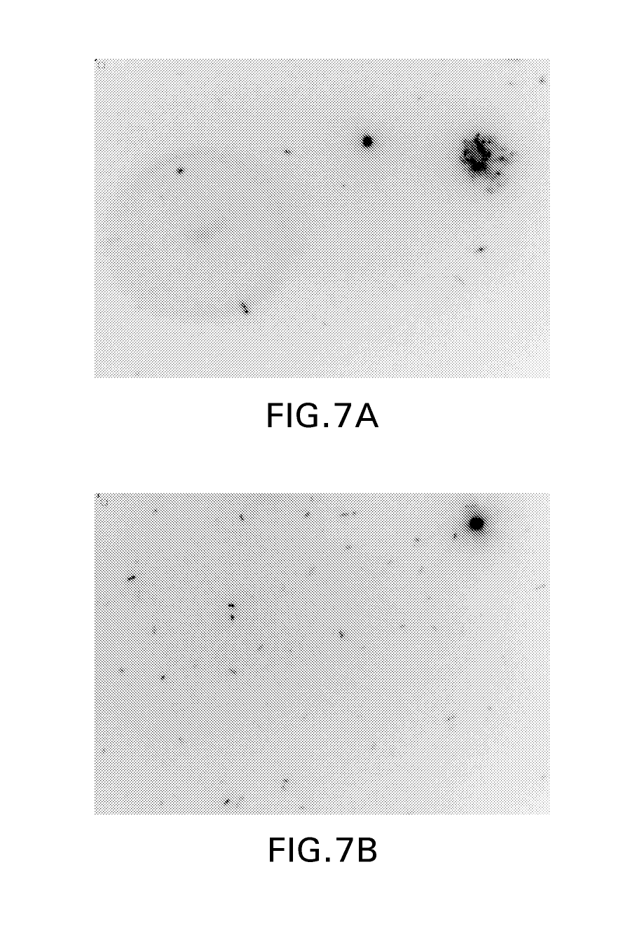

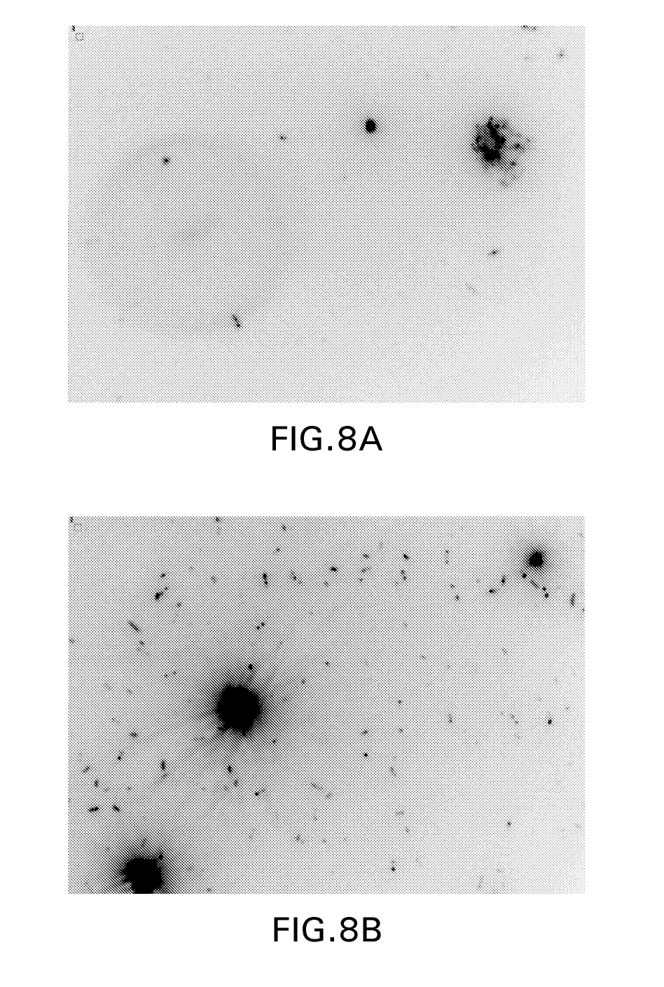

Polyclonal antibodies raised against loop 1 and loop 2 were tested for their ability to detect OprM on a whole P. aeruginosa cell. Two cell lines were used in the assay—6477 that expresses OprM and 6476 that does not express OprM (as a negative control). Briefly, the cells in mid-log growth were pelleted by centrifugation and resuspended in blocking buffer (3% heat-inactivated fetal bovine serum in PBS) and blocked for 30 min at 37° C. Cells were pelleted and resuspended in primary polyclonal antibodies at various dilutions in PBS (1:100 dilution was used in FIGS. 7 and 8). Antibodies were allowed to bind for 45 min. at 37° C. Cells were washed 2× in PBS. Fluorescently labeled secondary antibody (goat anti-rabbit IgG Alexa-488) was used at a 1:200 dilution in PBS. Secondary antibody was incubated for 45 min. at 37° C. Cells were washed 2× in PBS, dispensed into a 384 well Greiner imaging plate. Images were acquired utilizing the BD Pathway 435 automated imaging platform and a 60× objective.

Images from the Alexa 488 channels are shown in FIG. 7 (for polyclonal raised against loop 1 in rabbit #3) and FIG. 8 (for polyclonal raised against loop 2 in rabbit #5).

### Example 6

### Detection of Pseudomonas Proteins in Urine

In order to be useful as a diagnostic, OprM must be detectable in biological samples from infected patients. Experiments were conducted to assay for OprM in biological samples as well as to determine the ability of shotgun sequencing with liquid chromatography coupled with tandem mass spectroscopy (LC-MS/MS) to identify Pseudomonas peptides in urine.

The limit of detection of bacterial proteins that can be detected in human urine using shotgun sequencing (LC-MS/MS) was determined in two ways: (1) by detecting three Pseudomonas protein controls that had been spiked into urine proteins and (2) by detecting excreted protein extracts from Pseudomonas grown in various culture conditions spiked in at different dilutions in to human urine samples. Both in-solution and in-gel (SDS PAGE) digestion methods followed by shotgun sequencing LC-MS/MS were evaluated to identify bacterial protein in human urine (see, e.g., Spahr et al., 2001, Proteomics 1:93-107). Urine without bacterial proteins spiked in were used as negative controls.

Detection of Bacterial Proteins in Urine

Urine samples were spun down to removed particles and then were concentrated using a molecular cutoff filtration device (Amicon 3K Ultra Centrifuge Filter; Millipore, Billerica, Mass.). The filtration was repeated once using ammonium bicarbonate for buffer exchange. Protein concentration of retentate was measured by BCA assay.

Three P. aeruginosa protein controls (Lectin 100 fmol/μl; Azurin 10 fmol/μl; Exotoxin 1 fmol/μl) were spiked into 20 μg total urine proteins. An in-solution digestion was used to hydrolyze the proteins present in the sample into peptides in preparation for analysis by shotgun LC-MS/MS.

10 μl of 100 mM DTT in 50 mM ammonium bicarbonate was added to the spiked urine sample. 50 mM ammonium bicarbonate was then added to a final volume of 180 μl and incubated at 60° C. for 30 min. After incubation, 10 μl of a 300 mM iodoacetamide in 50 mM ammonium bicarbonate solution as added and incubated at room temperature for 30 min. After incubation, add 10 μl of 0.05 trypsin solution to the sample and incubate at 37° C. for 12-16 hours.

The liquid chromatography portion of the shotgun LC-MS/MS method was preformed by eluted the peptides from a C18 nano-spray column using a gradient from 2% to 70% Solvent B (0.1% TFA in acetonitrile).

The tandem mass spectrometry portion of the shotgun LC-MS/MS method was performed by detecting the peptides using data-dependent acquisition mode in the LTQ-Orbitrap spectrophotometer (Thermo Electron Corp., Rochester, N.Y.). Ions with m/z from 300 to 2000 were scanned in orbitrap with resolution of 60000. The top 20 ions with highest intensity in each scan were fragmented to generate MSMS spectra for peptide identification and database search.

The shotgun LC-MS/MS analysis revealed one peptide of Lectin (added at 100 fmol/μl) and 1 peptide of Azurin (10 fmol/μl) suggesting that the limit of detection for the standards used was 10-100 fmol/μl for in-solution digested samples. Thus, this methodology can be used to determine if OprM is detectable as an excreted protein in P. aeruginosa cultures.

OprM in Culture Supernatant

Studies characterizing the proteins excreted by P. aeruginosa into the culture media were performed using four strains of P. aeruginosa (CLB 2428, CL5701, CLB24388 and PAO-1). All four strains grown in both M9 and MH2 media. There was no significant background level of contaminating protein in any of the culture media. The supernatants were collected and the excreted proteins were isolated. Both in-solution and gel based (SDS PAGE) digestion approaches were employed to prepare the samples for shotgun LC-MS/MS so that bacterial proteins could be identified in the samples.

The in-solution digestion was performed as described supra. The in-gel trypsin digestion was performed as follows. PAGE gel bands were cut into 1:1 mm pieces on a microscope slide and placed into a 1.5 ml Eppendorf tube. If the gel had been stained, the gel slices were destained by immersion in approximately 400 μl of a 25 mM ammonium bicarbonate/30% acetonitrile solution for 10 minutes. The solution was removed using a pipette and discarded. The process was repeated until the gel slice was clear.

The gel slices were dehydrated by adding 400 μL of 100% acetonitrile until the gel slices shrunk and became an opaque-white color. The aceronitrile was removed and let air-dry for 5-10 minutes.

100 μl of a 0.15 mM DTT solution was added to each sample and incubated at 60° C. for 30 min. The liquid was then removed before washing with acetonitrile until the gel slices had fully shrunk. The acetonitrile was removed before the gel slices were air dried.

100 μl of a 0.3 mM ammonium bicarbonate solution was added to each gel slice and incubated at room temperature 15 min. in the dark. The liquid was then removed before washing with acetonitrile until the gel slices had fully shrunk. The acetonitrile was removed before the gel slices were air dried.

50 μl of a 12.5 ng/μl trypsin solution was added to each gel slice and incubated on ice for 30 min. The gel slices were then incubated overnight at 37° C. After the incubation, the liquid was transferred to a 0.5 ml Eppendorf tube with care taken to not transfer the gel slices. 200 μl of a 50% acetonitrile/5% acetic acid solution was added and incubated at room temperature for 20 min. The samples were then subjected to a speed vac with low heat to near dryness and stored at −20° C. until analysis.

Using in-solution digestion of excreted proteins followed by LC-MS/MS, 426 unique proteins were identified. Using an in-gel based approach followed by LC-MS/MS, 912 proteins were identified. A total of 1157 unique P. aeruginosa proteins were identified using 4 bacterial strains grown in MH2 and M9 media.

OprM was identified using both methods. This indicated that OprM is a good candidate for infection detection using patent biological samples as it will likely be shed into the sample.

The supernatant proteins were also used to spike into urine samples to determine if OprM could be detected in those conditions. The supernatant proteins were spiked in to human urine at 10:1, 100:1 and 1000:1 (human urine proteins:bacteria proteins). Pseudomonas proteins were identified in both in-solution and in-gel digested samples even when mixed at 1000:1 human urine proteins:bacteria proteins.

Other embodiments are within the following claims. While several embodiments have been shown and described, various modifications may be made without departing from the spirit and scope of the present invention.

1. A composition comprising an immunologically effective amount of one or more polypeptides comprising an outer loop of Pseudomonas aeruginosa (P. aeruginosa) OprM and a pharmaceutically acceptable carrier.

2. The composition of claim 1, wherein the OprM is SEQ ID NO:5.

3. The composition of claim 2, wherein the one or more polypeptides comprise an outer loop selected from the group consisting of SEQ ID NOs:1 and 2.

4. The composition of claim 3, wherein the one or more polypeptides further comprise up to 10 amino acid residues that are adjacent to the outer loop.

5. The composition of claim 4, wherein the one or more one or more polypeptides comprise SEQ ID NOs:3 or 4.

6. The composition of claim 5, wherein the polypeptides comprising an OprM outer loop is conjugated to a carrier protein.

7. The composition of claim 6, wherein the carrier protein is KLH.

8. The composition of claim 5 wherein the composition further comprises an adjuvant.

9. A method of inducing a protective immune response in a patient against a P. aeruginosa infection comprising the steps of administering to the patient an immunologically effective amount of the composition of claim 3.

10. The method of claim 9, wherein the patient is human.

11. The method of claim 10, wherein the patient is selected from the group consisting of cystic fibrosis patients, burn patients, hospital patients, patients undergoing surgery, patients on ventilators and patients with weakened immunity.

12-18. (canceled)

19. A method of conferring passive immunity to a P. aeruginosa infection in a patient comprising administering to the patient one or more antibodies that specifically bind to an outer loop of a P. aeruginosa OprM.

20. The method of claim 19 wherein the one or more antibodies are monoclonal antibodies.

21. The method of claim 20, wherein the one or more antibodies are selected from the group consisting of human antibodies and humanized antibodies.

22. The method of claim 21, wherein the one or more outer loops are selected from the group consisting of SEQ ID NOs:1 and 2.

23. A method of detecting a P. aeruginosa infection in a patient comprising:
a) collecting a biological sample from the patient;
b) contacting the biological sample with an antibody that binds to an outer loop of a P. aeruginosa OprM under conditions that allow for immunocomplex formation;
c) detecting binding of the antibody to the biological sample, wherein binding indicates presence of a P. aeruginosa infection in the patient.

24. The method of claim 23, wherein the biological sample is selected from the group consisting of sputum, endotracheal aspirate, bronchiolar levage fluid, respiratory tract sample, urine, blood, plasma, saliva, pus, ascites, wound exudate, peritoneal fluid, abdominal fluid and tears.

25. The method of claim 23, wherein the OprM is SEQ ID NO:5.

26. The method of claim 25, wherein the outer loop is selected from the group consisting of SEQ ID NOs:1 and 2.

27. The method of claim 26, wherein the antibody is a polyclonal or monoclonal antibody.
